(19) United States
(12) Patent Application Publication
(10) Pub. No.: US 2019/0373870 A1
(43) Pub. Date: Dec. 12, 2019

(54) BENEFICIAL YEASTS FOR ARTHROPODS

(71) Applicants: BIOBEST GROUP N.V., Westerlo (BE); KATHOLIEKE UNIVERSITEIT LEUVEN, Leuven (BE)

(72) Inventors: Maria Isabel Pozo Romero, Begijnendijk (BE); Alfredo Benavente Martinez, Begijnendijk (BE); Annette Van Oystaeyen, Landorp (BE); Hans Jacquemyn, Bierbeek (BE); Jacek Bartlewicz, Gdansk (PL); Felix Wäckers, Antwerpen (BE)

(73) Assignees: BIOBEST GROUP N.V., Westerlo (BE); KATHOLIEKE UNIVERSITEIT LEUVEN, Leuven (BE)

(21) Appl. No.: 16/462,963

(22) PCT Filed: Dec. 1, 2017
(86) PCT No.: PCT/EP2017/081095

§ 371 Date: May 22, 2019

(30) Foreign Application Priority Data
Dec. 1, 2016 (NL) 2017905

(51) Int. Cl. A01K 67/033; A01K 53/00; C12R 1/645; A61K 36/064; A23K 20/163; A23K 10/16; A23K 20/174; A23K 20/158; A23K 20/20

(52) U.S. Cl. CPC A01K 67/033; A01K 53/00; C12R 1/645; A61K 36/064; A23K 20/20; A23K 10/16; A23K 20/174; A23K 20/158; A23K 20/163

(57) ABSTRACT

Methods for rearing arthropods, particularly pollinating insects, comprising providing said arthropods with a Wickerhamiella yeast, preferably Wickerhamiella bombiphila yeast, also known as Candida bombiphila, or substances produced thereby, particularly comprised within a food composition, are provided herein. Advantageously, said yeast, particularly food compositions comprising said yeast, when provided to an arthropod, particularly a pollinating insect, improves the fitness, health, behaviour and activity of the arthropod.

### TECHNICAL FIELD

The present invention provides methods of rearing arthropods and/or improving arthropod fitness, health and/or behavior, and the invention further provides food or feed compositions for use in such methods.

### BACKGROUND OF THE INVENTION

Many arthropods have important roles in the environment and are crucial to humans in many ways. For example, arthropods can be used in biological pest control, as decomposers or in the production of several human-made products such as wax, silk or ingredients for medications. Furthermore, several arthropods, such as crustaceans (e.g. shrimp, prawns, crabs, lobsters) and insects are cultivated for use as human food. However, the greatest contribution of arthropods to the human food supply is by providing pollination services, thereby ensuring the successful production of arthropod pollinated fruit-bearing crops.

Arthropod pollinators, in particular insects, play an important role in plant reproduction and ecosystem functioning, by providing plants with the benefits of crosspollination. Insects are the prime pollinators of most agricultural crops and wild plants. In entomophilous plants (comprising 87% of angiosperms), pollination by insects has been shown to improve crop yield, individual fruit quality and quantity, shelf-life, taste, nutritional composition and market value compared to self-pollination. As a result, pollinator abundance and richness are essential features for both agricultural productivity and the conservation of wild plant communities. In turn, plants provide the visiting insects with nectar and pollen as the main floral rewards. However, in Western Europe and many other parts of the world, intensification of traditional farming practices over the last century has led to impoverished landscapes that represent poor habitats for many flower dependent insects due to a lack of suitable floral resources. In addition, chemical pesticides used in intensive agricultural production have been shown to have strong direct and indirect negative effects on pollinators. For example, fungicides may influence the gut flora of arthropods and thereby affect the host's health and/or ability to digest food. Finally, certain insect pollinators (bees) are susceptible to various diseases and pests (mites). It is believed that these three (often interacting) mechanisms are the main factors underlying the decline of insect pollinator communities worldwide.

Efforts are being made to counteract this trend and to sustain pollinator diversity and fitness. The health, behavior and numbers of the pollinating insects can be improved by, for example, increasing the quantity and quality of their habitats, increasing the public awareness, prohibiting use of pollinator-harming pesticides, supporting bee keeping, etc. However, the need remains to improve pollinator fitness and health in order to ensure future pollination of both cultivated crops and wild vegetation.

Arthropods also play an important role in agriculture as predators and parasitoids in biological pest control. Also in this context improvement of biodiversity is important, as species-rich populations are more likely to control pests than poorer ones.

Thus, there is a general need for improving methods which allow improvement of fitness and health of beneficial arthropods.

### SUMMARY OF THE INVENTION

Described herein are methods for rearing arthropods, particularly pollinating insects, and/or improving their fitness, health and/or behaviour, by providing the arthropods with a Wickerhamiella yeast, particularly a Wickerhamiella bombiphila yeast—previously also known as Candida bombiphila, such as specific Wickerhamiella bombiphila (Candida bombiphila) strains and/or fragments thereof or substances produced by said yeast. Described herein are also various means for providing the arthropods, particularly pollinating insects, with said Wickerhamiella yeast, particularly Wickerhamiella bombiphila (Candida bombiphila) yeast, such as in particular via a food or feed composition, comprising said yeast or substances produced by said yeast. The different aspects and embodiments of the present invention advantageously alleviate some of the problems of the prior art. In particular, providing a Wickerhamiella yeast, particularly Wickerhamiella bombiphila (Candida bombiphila) yeast to arthropods particularly pollinating insects, was found to improve the fitness, health and/or behaviour of these arthropods, resulting in more robust and healthy arthropod populations. Pollinator communities with increased fitness contribute to increased pollination activity and, subsequently, ensure the successful production of pollinated fruit-bearing crops and the reproduction of wild vegetation.

The application thus provides methods for rearing arthropods and/or improving the fitness of arthropods, comprising providing said arthropods with a Wickerhamiella yeast, particularly Wickerhamiella bombiphila (Candida bombiphila), fragments thereof or substances produced thereby. In particular embodiments, said arthropods are colony-forming arthropods. More particularly, said methods are methods for improving the development, size and/or fitness of a colony of arthropods.

In particular embodiments of the different methods as envisaged herein, said arthropods are provided with a Wickerhamiella bombiphila (Candida bombiphila) yeast material selected from:

(i) living cells of said Wickerhamiella yeast;

(ii) dead cells of said Wickerhamiella yeast; or

(iii) a composition comprising a growth medium in which said Wickerhamiella yeast has been inoculated and cultivated. In particular embodiments, said growth medium comprises living cells of said yeast and/or dead cells of said yeast. In further embodiments, said growth medium is a medium from which the (living or inactivated) yeast cells have been removed after having been cultivated therein. Thus, in particular embodiments, the growth medium no longer comprises yeast cells but comprises substances produced by said Wickerhamiella yeast during cultivation on the medium and/or comprises yeast fragments.

In particular embodiments, the application provides methods for cultivating a fruit-bearing crop which involve pollinating the flowering crop by an arthropod, wherein the arthropod has been provided with a Wickerhamiella yeast, particularly Wickerhamiella bombiphila (Candida bombiphila), fragments thereof or substances produced thereby. More particularly, the methods comprise the steps of providing the flowering fruit-bearing crop, providing a pollinating arthropod to said crop, wherein said arthropod is reared according to the methods described herein and allowing pollination of the flowering crop by the pollinating arthropod.

The application further provides the use of a Wickerhamiella yeast, particularly Wickerhamiella bombiphila (Candida bombiphila), fragments thereof or substances produced thereby for improving or enhancing the health, fitness and/or behaviour of arthropods. In particular embodiments, said Wickerhamiella yeast, particularly Wickerhamiella bombiphila (Candida bombiphila) decreases gut parasites such as Crithidia bombi.

In particular embodiments of the uses provided herein, the arthropods are pollinating flying insects. In particular embodiments, the Wickerhamiella yeast, particularly Wickerhamiella bombiphila (Candida bombiphila), fragments thereof or substances produced thereby are used to improve flight activity of said arthropods.

In particular embodiments of the methods and uses described herein, said Wickerhamiella yeast, particularly Wickerhamiella bombiphila (Candida bombiphila), fragments thereof or substances produced thereby are comprised within a food composition as also described herein.

In particular embodiments of the methods and uses provided herein said Wickerhamiella bombiphila (Candida bombiphila) is a Wickerhamiella bombiphila/Candida bombiphila strain deposited under the accession number MUCL 56142 at the BCCM/LMG culture collection.

The application further provides food compositions for arthropods comprising a Wickerhamiella yeast, particularly Wickerhamiella bombiphila (Candida bombiphila). Preferably, said food composition for arthropods comprises sugar water and/or pollen, and a Wickerhamiella yeast, preferably Wickerhamiella bombiphila, fragments thereof or substances produced thereby. In particular embodiments, the food composition for arthropods as envisaged herein comprises sugar water and/or pollen and (i) living cells of said Wickerhamiella yeast;

(ii) dead cells of said Wickerhamiella yeast; or (iii) a growth medium in which said Wickerhamiella yeast was inoculated and wherein said growth medium comprises living cells of said yeast, or dead cells of said yeast or wherein the yeast cells have been removed from said growth medium following incubation of the growth medium inoculated with said Wickerhamiella yeast.

In particular embodiments, said food composition, in addition to the yeast cells or products derived therefrom comprise (i) a carbohydrate source, preferably a sugar, or nectar or honey or a substitute thereof; and (ii) optionally, one or more of the following diet components: a nitrogen source, vitamins, lipids or fats and/or minerals. In particular embodiments, the carbohydrate source is a sugar chosen from sucrose, glucose, maltose, dextrose, fructose, invert sugar, corn syrup or glucose syrup, and combinations thereof.

In particular embodiments of the methods, uses and food compositions provided herein, said arthropods are insects, preferably Hymenoptera. In further particular embodiments, said Hymenoptera are Apocrita, preferably Apoidea, more preferably bees or bumble bees.

The application further provides Wickerhamiella bombiphila/Candida bombiphila strains which are particularly suitable for the methods and uses provided herein. In a more particular embodiment, the strain is the strain deposited under the accession number MUCL 56142 at the BCCM/LMG culture collection, or variants thereof.

### BRIEF DESCRIPTION OF THE FIGURES

The following description of the figures of specific embodiments of the invention is merely exemplary in nature and is not intended to limit the present teachings, their application or uses.

FIG. 1. Suppression of the bumblebee pathogen Crithidia bombi by live cells of the yeast Wickerhamiella bombiphila (Candida bombiphila) according to a particular embodiment of the invention. The proportion of live cells is shown for the two tested species under two different atmospheric conditions. The triangles show the mean proportion of live cells of the gut parasite Crithidia bombi and circles indicate the mean proportion of live cells of the yeast Wickerhamiella bombiphila (Candida bombiphila). The proportion of live cells is shown for the single-species controls (white shapes) and for the two-species mixes (full black shapes) with bars indicating the standard errors. Different letters indicate significantly different results with P values calculated based on the least squares means of the generalized linear model.

FIG. 2. Impact of the yeast Wickerhamiella bombiphila (Candida bombiphila) on flight activity of treated bumblebee colonies. The model-adjusted mean number of workers flying in and out of the hive per 5-minute counting round for the control colonies (black) and Wickerhamiella bombiphila (Candida bombiphila) treated colonies (white). The sum of both in- and out-flying workers is given, as well as total flight activity. Error bars indicate the standard errors. P and Z values are calculated based on least squares means of a generalized linear mixed model.

FIG. 3. Model-adjusted mean number of workers and future workers (=sum of pupae and workers) for both the control treatment (black) and Wickerhamiella bombiphila (Candida bombiphila) treatment (white), counted after a period of eight weeks. Error bars indicate the standard errors. P and Z values are calculated based on least squares means of a generalized linear model.

FIG. 4. The model-adjusted mean rate of weekly increase in the full colony size (sum of all developmental stages), the amount of brood (eggs and larvae), and the future workers (sum of pupae and emerged workers) is shown for the control treatment (black) and the Wickerhamiella bombiphila (Candida bombiphila) (Cbh) treatment (white). Asterisks show significance levels based on P values of a generalized linear mixed model (*=P<0.05).

FIG. 5. Mean number of days to reach the different developmental stages (eggs, pupae and emerging adult (workers)) are shown for the control treatment (black) and the Wickerhamiella bombiphila (Candida bombiphila) (Cbh) treatment (white). The dotted lines represent the standard errors.

FIG. 6. The model-adjusted mean number of dead larvae summated over the whole experiment (12 weeks) is shown for the control treatment (black) and the Wickerhamiella bombiphila (Candida bombiphila) treatment (white). Error bars indicate the standard errors. P and Z values are calculated based on least squares means of a generalized linear model.

FIG. 7. Model-adjusted mean number of produced sexuals (males and queens) in the control group (black) and the Wickerhamiella bombiphila (Candida bombiphila) treatment group (white). Error bars indicate the standard errors. P and Z values are calculated based on least squares means of a generalized linear model.

FIG. 8. Effect of a MUCL 56142 strain (named the “Biobest strain”) and the “type strain” CBS 9712T on colony development, compared to a control treatment that did not contain any yeasts in the sugar water according to an embodiment of the invention.

FIG. 9. Brood development (left) and number of predicted workers (right) after week 5 for colonies fed with control compositions (black bar) or C. bombiphila supplemented pollen (white bar). Bar height indicates model-adjusted average+−SE. Different letters denote means that are different at p<0.05, for a given variable.

FIG. 10. Number of workers (left) and number of males at week 8 (right) for colonies fed with control, unsupplemented pollen (black bar) and C. bombiphila supplemented pollen bread (white bar). Bar height indicate model-adjusted average+−SE. Different letters denote means that are different at p<0.05, for a given variable.

FIG. 11. Model-adjusted mean+−SE of predicted workers (sum of pupae and emerged workers) for the control treatment (black) and the 4 C. bombiphila (Cbh) treatments [from left to right: active yeast cells (treatment 1); yeast cells which have been inactivated after 3 days (treatment 2); inactivated yeast cells isolated from the growth medium added to the sugar water (treatment 3); growth medium wherein the yeast cells have been inactivated after 3 days & filtered (treatment 4)] for week 5 (upper panel) and week 10 (lower panel). Different letters above bars denote significant differences at P<.0.05.

FIG. 12. Average number of days +−SE before the emergence of the first worker in control (black bar) vs C. bombiphila-treated colonies. Different bars denote different C. bombiphila administration treatments [from left to right: active yeast cells (treatment 1); yeast cells which have been inactivated after 3 days (treatment 2); inactivated yeast cells isolated from the growth medium added to the sugar water (treatment 3); growth medium wherein the yeast cells have been inactivated after 3 days & filtered (treatment 4)]. Dash line denotes the fastest appearance of workers in baseline, control conditions.

FIG. 13. Female fitness (sum of queens and workers) produced per colony (mean+−SE) after a 16-week-period in control (black bar) vs C. bombiphila-treated colonies. Different bars denote different C. bombiphila administration treatments (from left to right: active yeast cells (treatment 1); yeast cells which have been inactivated after 3 days (treatment 2); inactivated yeast cells isolated from the growth medium added to the sugar water (treatment 3); growth medium wherein the yeast cells have been inactivated after 3 days & filtered (treatment 4)]. Different letters denote means that were significantly different at P<0.05.

FIG. 14. Flight activity (sum of incoming and outgoing bees per 5 min census) of C. bombiphila treated colonies compared to control colonies (black bar), after 1 and 2 weeks after colony placement in an apple orchard in Sint-Truiden, Belgium. Different bars denote different C. bombiphila administration treatments (from left to right: active yeast cells (treatment 1); yeast cells which have been inactivated after 3 days (treatment 2); inactivated yeast cells isolated from the growth medium added to the sugar water (treatment 3); growth medium wherein the yeast cells have been inactivated after 3 days & filtered (treatment 4)]. Different letters denote means that were significantly different at P<0.05.

FIG. 15. Dual-choice behavioral test performed using a colony of naïve B. terrestris workers and 16 artificial flowers containing either control sugar water (black bar, no yeasts) or sugar water comprising living C. bombiphila cells (white bar). Preference was evaluated by recording the total number of visits to each treatment (left) or the probing time that bumblebees spent, on average, on those flowers (right).

### DETAILED DESCRIPTION OF THE INVENTION

It is to be understood that the terminology used herein is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein may be used in the practice or testing of the present invention, the preferred methods and materials are now described.

While potentially serving as a guide for understanding, any reference signs in the claims shall not be construed as limiting the scope thereof.

In this specification and the appended claims, the singular forms “a”, “an”, and “the” include plural references unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”. The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

In the following passages, different aspects or embodiments of the invention are described in more detail. Each aspect or embodiment so described may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification may but need not necessarily refer to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The terms “Candida bombiphila” and “Wickerhamiella bombiphila” as used herein are used interchangeably, and refer to the same yeast species. In fact, two strains of this species were first described in 2004 by Brysch-Herzberg and the closest relative of the species was found to be Wickerhamiella domerquiae (Herzberg and Lachance, 2004, International Journal of Systematic and Evolutionary Microbiology, 54: 1857-1859). As, at the time, these authors could not conjugate them to allow sexual reproduction, the species was described as an asexual clade, Candida, nomenclature that was originally used to indicate imperfect or asexual yeasts and has been applied to highly divergent species. Recent research based on the DNA-based phylogeny of the species suggests that the species formerly known as “Candida bombiphila” should now be renamed as Wickerhamiella bombiphila (de Vega et al, 2017, FEMS Yeast Research, Volume 17, Issue 5, 1 Aug. 2017).

The inventors have surprisingly found that certain yeasts are able to improve various aspects of the general fitness of an organism, in particular an arthropod, when provided therewith, more particularly when the yeast is ingested by said arthropod. In particular, the inventors have found that Wickerhamiella bombiphila (Candida bombiphila) or fragments thereof or substances produced thereby, when provided to an arthropod such as in a sugar solution or via pollen, enhances rearing, health and/or behaviour and improves the fitness of the arthropod. Indeed, the inventors have found that providing Wickerhamiella bombiphila (Candida bombiphila) to bumblebees, in particular via a food composition (sugar solution and/or pollen) comprising said yeast, increases the size, the brood, the number of workers, the number of male sexuals and/or the number of predicted workers of a colony of bumblebees and/or decreases the number of dead larvae. Furthermore, the inventors have isolated a particular strain of Wickerhamiella bombiphila (Candida bombiphila) which has been shown to be particularly potent in evoking the effects described above when provided to an arthropod.

In the context of the different aspects and embodiments of the present invention, the term “arthropod” as referred herein can be any arthropod from the phylum Arthropoda, including insects, arachnids, myriapods and crustaceans. Preferably, the arthropods are arthropods important as feed or food for animals, such as livestock, dairy animals, fish and/or humans, or are arthropods providing other products, such as silk, or services such as pollination and biological pest control. In particular embodiments, the arthropods are insects, preferably pollinating insects. Non limiting examples of pollinating insects are bees, butterflies, moths, ants, wasps, flies, midges, mosquitoes or beetles. In particular embodiments, the arthropods are bees, preferably, bumblebees or honey bees, more preferably bumblebees of the genus Bombus. In particular embodiments, the arthropods are pollinating colony-forming insects. In particular embodiments, the insects belong to the order of Hymenoptera, such as to the suborder of Apocrita, more particularly to the superfamily of Apoidea. In particular embodiments, the arthropods belong to the family of Apidae. In further particular embodiments, the insects are from the genus Acamptopeum, Anthemurgus, Antherenoides, Acanthopus, Afromelecta, Agapanthinus, Aglae, Aglaomelissa, Alepidosceles, Alloscirtetica, Amegilla, Ancyla, Ancyloscelis, Anthophora, Anthophorula, Apis, Apotrigona, Arhysoceble, Austroplebeia, Axestotrigona, Bombus, Brachymelecta, Caenonomada, Camargoia, Canephorula, Cemolobus, Centris, Cephalotrigona, Chalepogenus, Chilamalopsis, Cleptotrigona, Coelioxoides, Ctenioschelus, Ctenoplectra, Ctenoplectrina, Cubitalia, Dactylurina, Deltoptila, Diadasia, Diadasina, Duckeola, Elaphrophoda, Epeoloides, Epicharis, Epiclopus, Eremapis, Ericrocis, Eucera, Eucerinoda, Eufriesea, Euglossa, Eulaema, Exaerete, Exomalopsis, Florilegus, Friesella, Frieseomelitta, Gaesischia, Gaesochira, Geniotrigona, Geotrigona, Habrophorula, Habropoda, Hamatothrix, Heterotrigona, Homotrigona, Hopliphora, Hypotrigona, Isepeolus, Lanthanomelissa, Leiopodus, Lepidotrigona, Lestrimelitta, Leurotrigona, Liotrigona, Lisotrigona, Lophothygater, Lophotrigona, Martinapis, Melecta, Melectoides, Meliphilopsis, Meliplebeia, Melipona, Meliponula, Melissodes, Melissoptila, Melitoma, Melitomella, Meliwilea, Mesocheira, Mesonychium, Mesoplia, Micronychapsis, Mirnapis, Monoeca, Mourella, Nannotrigona, Nanorhathymus, Nogueirapis, Notolonia, Odontotrigona, Osirinus, Oxytrigona, Pachymelus, Pachysvastra, Papuatrigona, Paratetrapedia, Paratrigona, Paratrigonoides, Paepeolus, Pariotrigona, Partamona, Peponapsis, Platysvastra, Platytrigona, Plebeia, Plebeiella, Plebeina, Protosiris, Ptilothrix, Ptilotrigona, Rhathymus, Santioga, Scaptotrigona, Scaura, Schwarziana, Schwarzula, Simanthedon, Sinomelecta, Sundatrigona, Svastra, Svastrides, Svastrina, Syntrichalonia, Tapinotapsis, Tapinotaspoides, Tarsalia, Teratognatha, Tetragona, Tetragonilla, Tetragonisca, Tetragonula, TetraIonia, Tetraloniella, Tetralonioidella, Tetrapedia, Tetrigona, Thygater, Thyreomelecta, Thyreus, Toromelissa, Trichocerapis,

Trichotrigona, Trigona, Trigonisca, Trigonopedia, Ulugombakia, Xenoglossa, Xeromelecta, Zacosmia, Aethammobates, Ammobates, Biastes, Brachynomada, Caenoprosopina, Caenoprosopis, Chiasmognathus, Doeringiella, Epeolus, Hexepeolus, Holcopasites, Kelita, Melanempis, Neolarra, Neopasites, Nomada, Odyneropsis, Oreopasites, Parammobatodes, Paranomada, Pasites, Pseudepeolus, Rhinepeolus, Rhogepeolus, Rhopalolemma, Schmiedeknectia, Sphecodopsis, Spinopasites, Thalestria, Townsendiella, Triepeolus, Triopasites, Allodape, Allodapula, Braunsapis, Creatina, Compsomefissa, Effractapis, Eucondylops, Exoneura, Exoneurfia, Exoneuridia, Macrogalea, Manuelia, Nasutapis, or Xylocopa. Preferably, the arthropods are bees or bumblebees, in particular bumblebees of the genus Bombus, such as B. terrestris, B. ignitus, B. diversus, B. occidentalis, including related species and sub-species. In particular embodiments, the arthropod is B. terrestris; such as B. terrestris africanus, B. terrestris audax, B. terrestris calabricus, B. terrestris canariensis, B. terrestris dalmatinus, B. terrestris lusitanicus, B. terrestris sassaricus, B. terrestris terrestris and B. terrestris xanthopus. In other embodiments said arthropod is a biological control agent for the biological control of pests, as known by the skilled person, such as a predatory mite, a parasitic wasp or a predatory insect, such as a ladybug, hoverfly, lacewing or a Mirid bug.

Provided herein are methods for rearing arthropods and/or for improving the health, behaviour and/or fitness of arthropods, comprising providing the arthropods with a Wickerhamiella yeast, particularly Wickerhamiella bombiphila (Candida bombiphila) yeast, fragments thereof or substances produced thereby. Provided herein is also the use of a Wickerhamiella yeast, particularly Wickerhamiella bombiphila (Candida bombiphila) yeast, fragments thereof or substances produced thereby, for rearing arthropods or for improving the health, behaviour and/or fitness of said arthropods.

In contrast to what its name may imply, the yeast species Wickerhamiella bombiphila (Candida bombiphila) is quite uncommon in wild bumblebees. Brysch-Herzberg (FEMS Microbiology Ecology 50 (2004) 87-100) performed an extensive study on the yeast community involved in plant-bumblebee mutualism by analyzing 43 queens and 92 workers, as well as 9 honey pots and hundreds of flower and nectar samples. While certain yeasts, like Metschnikowia reukaufii, were found in the majority of bumblebee samples analyzed C. bombiphila yeasts were only isolated once or twice from bumblebee honey. This rather weak association was the basis for the naming of this yeast species. Consequently the author argued that no conclusion could be drawn about its ecology. To date there was no evidence that the species might inhabit the tongue of the bumblebee and consequentially could colonize nectar, as extensive, worldwide studies were not able to isolate Candida bombiphila from bumblebees. Additionally, as detailed inter alia in Experiment 1, the inventors have shown that W. bombiphila/C. bombiphila is completely absent in commercially reared bumblebees from several companies and W. bombiphila/C. bombiphila is only occasionally transmitted from an inoculated source mother queen to nest mates. In view of the above very tenuous association between W. bombiphila/C. bombiphila and bumblebees, or any other arthropods, the finding that providing arthropods, and especially bumblebees, with Candida bombiphila improves the health, behavior and/or fitness of said arthropods is very surprising. In particular embodiments, the Wickerhamiella bombiphila (Candida bombiphila) yeast as envisaged herein is the Wickerhamiella bombiphila (Candida bombiphila) strain MUCL 56142, as further detailed below. The term “rearing” as used herein, in the context of rearing arthropods, broadly refers to breeding and supporting growth, development, maintenance, and reproduction of arthropods. The skilled person will understand that the rearing methods will differ for different arthropods. Suitable rearing or keeping methods are known to the skilled person. For example, bumblebees (e.g. B. terrestris) can be kept in a dark nest box under standard climatic conditions (28° C. and 60% relative humidity) and are typically fed ad libitum.

The term “providing to or with” as used in the context of the claimed methods broadly refers to making the Wickerhamiella yeast, particularly the Wickerhamiella bombiphila (Candida bombiphila) yeast, fragments thereof or substances produced thereby, available to the arthropod, for example by offering the Wickerhamiella yeast, particularly the Wickerhamiella bombiphila (Candida bombiphila) yeast, fragments thereof or substances produced thereby, as a food source or part of a diet. More particularly, the invention provides methods which involve actively offering the yeast, fragments thereof or substances produced thereby to the arthropod. This typically implies that the arthropods are offered compositions comprising at least about 100 cells per microliter up to a maximum of 60000 cells per microliter or the equivalent thereof in fragments or products derived therefrom. Typically, the number of yeast cells in the composition will naturally increase in the feeding solution. Offering the Wickerhamiella yeast, particularly the Wickerhamiella bombiphila (Candida bombiphila) yeast, fragments thereof or substances produced thereby, to the arthropod allows for transfer thereof to the arthropod, particularly the gut of the arthropod. The inventors have more particularly found that the effect of the Wickerhamiella yeast is not only ensured by living cells but can also be ensured by inactivated or dead cells, fragments thereof or substances produced thereby. Indeed, it has been found that a medium, in which Wickerhamiella yeast have been cultivated, is still capable of ensuring the desired effect on arthropods, even when the yeast cells have been removed from said medium. Accordingly, the effect can be ensured by providing the Wickerhamiella yeast in a direct manner, as living or dead cells, or in an indirect manner, via a medium in which said yeast cells have been cultivated (and from which they have been subsequently removed). In particular embodiments, the yeast cells may have been removed from the medium by filtration, such as by filtration with a filter with a pore diameter of 0.45 μm or less, such as 0.3 or 0.25 μm or less. Accordingly, in particular embodiments the Wickerhamiella bombiphila (Candida bombiphila) yeast as envisaged herein, fragments thereof or substances produced thereby, is provided to the arthropod as living cells, as dead cells or via a composition comprising a growth medium in which said Wickerhamiella yeast was inoculated and cultivated and wherein said growth medium comprises living cells of said yeast, or dead cells of said yeast or wherein said growth medium is a medium from which the (living or inactivated) yeast cells have been removed following incubation of the inoculated growth medium. Such a medium can thus be generated by simply cultivating Wickerhamiella yeast in growth medium and optionally removing the yeast cells therefrom, such as by filtration with a filter with a pore diameter of 0.45 μm or less, such as 0.3 or 0.25 μm or less. More particularly, in particular embodiments, the methods envisaged herein comprise the step of obtaining a growth medium comprising substances produced by Wickerhamiella yeast and/or fragments of said Wickerhamiella yeast as disclosed herein and providing said growth medium to said arthropods.

In particular embodiments, the Wickerhamiella yeast, particularly the Wickerhamiella bombiphila (Candida bombiphila) yeast as envisaged herein, fragments thereof or substances produced thereby, is provided in the form of a food composition, as further discussed below. In particular embodiments, the Wickerhamiella bombiphila (Candida bombiphila) strain MUCL 56142, as further detailed below, fragments thereof, or substances produced thereby, is/are provided to the arthropod. Alternatively, the yeast, yeast fragments or yeast substances can be provided to the arthropod by positioning them in the environment of the arthropod, so as to ensure direct contact of the arthropod therewith.

The term “Wickerhamiella yeast”, particularly the “Wickerhamiella bombiphila” (“Candida bombiphila”) when referred to as such or as used in the context of providing an arthropod with said yeast herein refers to either living yeast cells or inactivated, dead yeast cells of the said yeast. Preferably, the cells are living yeast cells.

The term “fragments” when referring to a yeast as used herein, refers to any component derived from a yeast cell. Non-limiting examples of such fragments are nucleic acids, proteins, peptides, polypeptides, the cell wall or a part of the cell wall or cell organelles. Such fragments can be provided as extracts, homogenates or isolated components.

The term “substances produced by a yeast” as used herein refers to the metabolites and other factors such as enzymes found in and/or produced by a yeast cell, such as by Wickerhamiella yeast, particularly the Wickerhamiella bombiphila (Candida bombiphila). For instance, the yeast metabolome database describes metabolites produced by the yeast Saccharomyces cerivisae. Similarly, non-Saccharomyces yeasts have been shown to produce enzymes such as pectinase, protease, glucanase, lichenase, β-glucosidase, cellulase, xylanase, amylase, sulphite reductase, and lipase (Jolly et al, 2006, S. Afr. J. EnoL Vitie., VoL 27, No.1: 15-39). Similar metabolites and/or enzymes can be identified for a Wickerhamiella yeast, particularly Wickerhamiella bombiphila (Candida bombiphila). These metabolites may be produced during fermentation. Non-limiting examples of metabolites include lipids, sterols, vitamins, amino acids, peptides, organic acids, sugars, glycoproteins or derivatives thereof, organic esters, higher aldehydes and alcohols, vicinal diketones (VDK), sulfur volatiles. In particular embodiments, the metabolites are glycoproteins. In further particular embodiments, the metabolites are “killer factors” in that they are toxic to other yeast strains and/or genera. In particular embodiments, the metabolites are provided as supernatant or extracts. In further embodiments, the metabolites are isolated metabolites. Preferably, the substances produced by the yeast as envisaged and/or fragments of the yeast as envisaged herein, may be provided as a growth medium in which Wickerhamiella yeast have been cultivated. As detailed above, such a medium is obtainable by allowing the Wickerhamiella yeast as envisaged herein to grow in suitable growth medium, and, subsequently, removing the yeast cells from said growth medium, such as by filtration, such as by filtration with a filter with a pore diameter of 0.45 μm or less, such as 0.3 or 0.25 μm or less, optionally after inactivation of the living yeast cells. Without wishing to be bound by theory, this way, a medium is obtained which comprises Wickerhamiella yeast metabolites and/or yeast fragments, but no Wickerhamiella yeast cells. Filters useful for removing yeast cells from growth media are known in the art.

In certain embodiments the invention provides methods for improving the health, fitness and/or behaviour of arthropods by providing the arthropods with a Wickerhamiella yeast, particularly a Wickerhamiella bombiphila (Candida bombiphila) yeast, preferably the Candida bombiphila strain MUCL 56142, fragments thereof or substances produced thereby, wherein said yeast is optionally comprised within a food composition as envisaged herein. In particular embodiments, said arthropods are pollinating and/or colony-forming insects, even more preferably bees or bumblebees. Without being bound by theory, it is believed that the yeast may provide certain components, such as decomposition products, and/or metabolites or substances, such as vitamins, sterols, or essential amino acids, to its arthropod host, which can in turn improve the health, fitness and/or behavior of the arthropod.

The term “health improvement”, as used in the context of the methods herein, refers to increasing the general well-being of the arthropod. The health of arthropods can be influenced by several factors acting in combination or separately. These include the effects of intensive agriculture and pesticide use, starvation, malnutrition, viruses, attacks by pathogens and internal or external parasites and environmental changes. A decreased health will most often also affect the behaviour of the arthropod in a negative manner. The health of an arthropod can be assessed by, inter alia, size, weight, lifespan, reproductive output and resistance to infection with pathogens and parasites. In particular, arthropods and especially bees seem to be particularly sensitive to gut parasites, such as Nosema bombi or Crithidia spp. In particular embodiments, the use of a Wickerhamiella yeast, particularly a Wickerhamiella bombiphila (Candida bombiphila) yeast, fragments thereof or substances produced thereby as envisaged herein, optionally comprised within a food composition as envisaged herein, improves the immune functioning of arthropods and reduces the number of gut parasites. Without being bound to theory it is believed that the impact on yeast on parasites and pathogenic bacteria and other fungi can be due to competition processes, and also due to priority effects: the changes (pH, carbon and nitrogen sources that are metabolized, produced by the 1st arriving organisms (i.e. yeast) make the environment unsuitable for later arriving organisms. Additionally or alternatively, it is known for some yeast species that they produce “killer factors”, i.e. proteins that are toxic for specific targets when they coexist. Hence, the present invention also relates to a method of improving immune functioning of arthropods, by providing said arthropods with a Wickerhamiella yeast, particularly a Wickerhamiella bombiphila (Candida bombiphila) yeast, preferably the Candida bombiphila strain MUCL 56142, fragments thereof or substances produced thereby, optionally comprised within a food composition as envisaged herein. Preferably, said arthropods are colony-forming arthropods, more preferably pollinating insects, even more preferably bees, most preferably bees of the genus Bombus. Advantageously, said method of improving immune functioning of arthropods leads to a decrease of gut parasites in arthropods, particularly a decrease of Crithidia bombi.

The term “fitness” as used herein when referring to an organism or group of organism is intended to refer to the ability of organisms or groups of organisms (e.g. colonies or populations) to survive and reproduce in the environment in which they find themselves. As a consequence of this survival and reproduction, the organism or group of organisms will contribute genes to the next generation (Orr, Nature Reviews. Genetics, 10 (2009) 531-539). Fitness comprises many different “fitness components” which contribute to the ability to produce viable progeny. As such, “fitness” also encompasses viability/longevity parameters, which are linked to general health and pathogen resistance but also mating success, and fecundity (daily fecundity/lifetime fecundity). In colony-forming species, such as B. terrestris, realised fecundity of the mother queen will be apparent in the number of individuals present in the colony. The term fitness may refer to physical or biological fitness, while both are usually linked to each other. Physical fitness is the physical ability to perform certain activities, such as flying, and may be evaluated by assessing the activity level, while biological fitness is the reproductive output, more particularly, the extent to which an organism is able to produce offspring in a particular environment. Both physical and biological fitness can inter alia be evaluated by assessing the colony development and/or colony developmental parameters as indicated above. The present invention thus also relates to a method of improving the biological, physical or both the biological and physical fitness of arthropods, by providing the arthropods with a Wickerhamiella yeast, particularly a Wickerhamiella bombiphila (Candida bombiphila) yeast, preferably the Candida bombiphila strain MUCL 56142, fragments thereof or substances produced thereby, optionally comprised within a food composition as envisaged herein. Preferably, said arthropods are colony-forming arthropods, more preferably pollinating insects, even more preferably bees, most preferably bees of the genus Bombus.

Certain embodiments of the present invention relate to methods of improving the behaviour or activity of an arthropod, particularly a pollinating insect, by providing said arthropod with a Wickerhamiella yeast, particularly a Wickerhamiella bombiphila (Candida bombiphila) yeast, preferably the Candida bombiphila strain MUCL 56142, fragments thereof or substances produced thereby, as provided herein, optionally comprised within a food composition as described herein. The behaviour of an arthropod, particularly a pollinating insect is apparent from typical activities performed by the arthropod or pollinating insect. These activities differ for different types of pollinating insects and between colony-forming or solitary pollinating insects. For example, central place foragers leave from their domicile/nest to go and search for food and return when they succeeded in their search. Accordingly, traffic leaving or returning to the colony can be used as a measure for the general behavior or activity of the pollinating insect. Alternatively, flight behaviour and foraging success can be used as a measure for the fitness of the pollinating (flying) insect, and, if the pollinating insect is a colony-forming insect, to study the colony fitness. A fit colony is characterised by a high number of pollinating insects making a high number of flights to gather food. An increase in flight activity and pollen/nectar collection will also lead to a more intense and a more efficient pollination of flowering crops. Non-limiting examples of other activities that can be studied to assess the behavior and fitness of colony-forming and/or pollinating insects are reaction to pheromones, temperature response, swarming behaviour, running behaviour, mating behaviour and/or frequency, grooming and/or hygienic behaviour, food storage behaviour, guarding behaviour and drifting behaviour.

In particular embodiments, said method of improving the behaviour or activity is a method for improving the flight and foraging activity of pollinating insects, preferably bees, more preferably bumblebees, even more preferably of bees of the genus Bombus, in particular B. terrestris. The term “flight activity” as used herein refers to the number of in- and out-flying pollinating arthropods and/or the frequency of in- and out-flying of pollinating arthropods from the nest over a certain period of time. The term “foraging activity” as used herein refers to the number of flowers visited per foraging flight and/or the amount of pollen or nectar collected.

In particular embodiments of the present invention, said arthropods are colony-forming arthropods and said Wickerhamiella yeast, particularly Wickerhamiella bombiphila (Candida bombiphila), fragments thereof, or substances produced thereby, optionally comprised within a food composition as further detailed below, improves colony development and/or the colony developmental rate. Accordingly, in particular embodiments, the present invention relates to methods for improving the colony development and/or the colony developmental rate of colony-forming arthropods, particularly pollinating colony-forming arthropods, comprising providing the arthropods with a Wickerhamiella yeast, particularly a Wickerhamiella bombiphila (Candida bombiphila) yeast, fragments thereof or substances produced thereby, preferably wherein said yeast is the Candida bombiphila yeast strain MUCL 56142, as discussed herein, wherein said Wickerhamiella yeast, particularly a Wickerhamiella bombiphila (Candida bombiphila) yeast is optionally comprised within a food composition as envisaged herein.

The term “colony development” as used herein refers to the advancement of a discrete group of arthropods, preferably eusocial arthropods, of the same species living and/or growing together in a shared domicile. More particularly, a colony of colony-forming arthropods is a highly-organised animal society with cooperative brood care, overlapping generations within a colony of adults and a division of labour/tasks into reproductive and non-reproductive groups. In colonies of certain arthropods, there is one queen or single breeding female who produces the offspring and many workers that take care of the eggs and larvae, forage for food and/or protect the colony. For example, a bee or bumblebee colony is initiated by a mother queen laying fertilised eggs in a nest. The queen starts a colony by laying fertilised eggs that will develop into workers that will feed the following groups of offspring and take care of foraging. As the colony grows progressively larger, male bees/bumblebees and new queens are produced as well. The term “colony developmental rate” refers to the speed at which the colony development occurs. Typically, the term “rate” as used herein refers to a rate of change, preferably per unit of time.

Colony development and/or colony developmental rate can be assessed by different parameters known by the person skilled in the art. For example, one could evaluate the time of first egg laying, the duration of development from egg to adult, or the number of individuals at a certain time interval, such as evaluating the size of the colony at time of sexual production, including evaluating all parameters relating to colony size, i.e. number of egg cups, larvae, pupae, workers, and sexuals (males and queens). The time interval or time of sexual production will differ depending on the developmental period of the type of colony-forming arthropod. An increase in the number of future workers, colony size and/or amount of brood within a certain time frame and/or when compared to a reference value is indicative of an improved colony development. Preferably, said reference value is the value obtained for a colony of Arthropoda not receiving the same treatment, i.e. not reared according to the methods according to present invention, or not having been provided with the yeast, fragment thereof or substances produced thereby as described herein. In certain embodiments, the present invention relates to a method for improving the size, the brood and/or the number of predicted workers of a colony of arthropods (compared to a reference colony) by providing the arthropods with a Wickerhamiella yeast, particularly a Wickerhamiella bombiphila (Candida bombiphila) yeast, fragments thereof or substances produced thereby, optionally comprised within a food composition according to the invention. Preferably said arthropods are colony-forming arthropods, more preferably pollinating insects, even more preferably bees, most preferably bees of the genus Bombus.

The application further provides agricultural methods which involve providing arthropods with yeast material as described herein. These methods include any methods which involves the use of beneficial arthropods. In particular embodiment, the method is a method of cultivating a crop which involves pollination and/or biological pest control by one or more arthropods.

As the pollination of flowering fruit-bearing crops is for a large part dependent on pollinating insects, the skilled person understands that the presence of lower numbers of pollinating insects, or less active or less fit/healthy pollinating insects has a negative effect on the pollination of insect pollinated crops, resulting in a decreased number of pollinated flowers, and consequently a decreased number of fruits which can be harvested. Accordingly, by providing a Wickerhamiella yeast, particularly a Wickerhamiella bombiphila (Candida bombiphila) yeast, preferably the Candida bombiphila strain MUCL 56142, fragments thereof or substances produced thereby, optionally comprised within a food composition as envisaged herein, to an arthropod, particularly a pollinating insect and thus increasing the health and/or fitness and/or behavior of the pollinating insect and/or (optionally) the colony development, the yield of fruit-bearing crops can be increased. Accordingly, the present invention further provides methods for cultivating a fruit-bearing crop, wherein a flowering crop is provided and wherein a flowering crop is pollinated by an arthropod, wherein said arthropods are reared according to the present invention or wherein said arthropods are provided with a Wickerhamiella yeast, particularly a Wickerhamiella bombiphila (Candida bombiphila) yeast, preferably the Candida bombiphila strain MUCL 56142, fragments thereof or substances produced thereby, optionally comprised within a food composition as provided herein. Preferably said arthropods are colony-forming arthropods, more preferably pollinating insects, even more preferably bees, most preferably bees of the genus Bombus.

In particular embodiments, the cultivation of the crop involves biological pest control by one or more arthropods. Where the biological control relies on the presence of arthropods, the skilled person understands that the presence of lower numbers of predatory arthropods, or less active or less fit/healthy predatory arthropods has a negative effect on biological pest control. Accordingly, the present invention further provides methods for cultivating a fruit-bearing crop, wherein arthropods are used to ensure biological pest control, and wherein said arthropods are reared according to the methods of the present invention.

The term “crop” as used herein refers to all cultivated plants or agricultural produce, grown for profit or subsistence. The term “fruit-bearing crop” as used herein refers to perennial edible crops, where the edible product is a true botanical fruit or is derived therefrom. The term “flowering crop” as used herein refers to flower-bearing crops, more particularly, crops which require pollination to enable fertilization. Non-limiting examples are apple, pear, quince, sorbus, loquat, cherry, plum, apricot, almond, peach, strawberry, raspberry, oleaster, sea buckthorn, European walnut, pecan, hazelnut, pistachio, olive, persimmon, fig, mulberry, pomegranate, feijoa, tangerine, orange, lemon, grapefruit, citron, currant, gooseberry, European hazel, Actinidia, Schizandra, honeysuckle, viburnum, barberry, avocado, date palm, mango, breadfruit tree, papaya, banana, tomato, peppers, melon, cucumber, squash, beans, cotton, and the like. The term “pollinate” or “pollination” as used herein refers to the process by which pollen is transferred to the female reproductive organs of a plant, thereby enabling fertilization to take place. For the process of pollination to be successful, a pollen grain produced by the anther, the male part of the flower, must be transferred to a stigma, the female part of the flower, of a plant of the same species. Pollination as referred herein is preferably cross-pollination, wherein the pollen from the anther of a flower on one plant is transferred to the stigma of the flower on another plant of the same species by a pollinating insect.

In particular embodiments, the application relates to methods which involve the arthropods directly, such as methods of pest control or methods of honey production. Indeed, the skilled person will understand that when the arthropods as referred to herein are honey bees, the methods for rearing honey bees, and/or the methods for improving the health, fitness, behaviour and/or activity of honey bees, such as increased flight activity described herein will lead to an increase in nectar collection and consequently an increase in honey production. Accordingly, the present invention also relates to methods for producing honey by providing honey bees with a Wickerhamiella yeast, particularly a Wickerhamiella bombiphila (Candida bombiphila) yeast, preferably the Candida bombiphila strain MUCL 56142, fragments thereof or substances produced thereby as envisaged herein, optionally comprised within a food composition as envisaged herein. In further embodiments, the application provides methods of biological pest control by providing predatory arthropods, such as mites, with a Wickerhamiella yeast, particularly a Wickerhamiella bombiphila (Candida bombiphila) yeast, fragments thereof or substances produced thereby.

In preferred embodiments of the present invention, the arthropods are provided with a Wickerhamiella yeast, particularly the Wickerhamiella bombiphila (Candida bombiphila) yeast, fragments thereof or substances produced thereby, ad libitum. The Wickerhamiella yeast, particularly the Wickerhamiella bombiphila (Candida bombiphila) yeast, fragments thereof or substances produced thereby, optionally comprised within a food composition as further described below, can be placed close to (e.g. inside or just outside of) the domicile/the nest of the arthropod or a natural feeding source of the arthropod and freely accessible to the arthropod. The nest can be either natural or artificial and where the arthropod is a colony-forming arthropod, the nest typically houses several generations of arthropods.

An easy and preferred means for providing an arthropod with a yeast as envisaged herein is via a food composition comprising said Wickerhamiella yeast, particularly said Wickerhamiella bombiphila (Candida bombiphila) yeast. Accordingly, the present invention further provides food or feed compositions for arthropods comprising a Wickerhamiella yeast, particularly a Wickerhamiella bombiphila (Candida bombiphila) yeast, preferably the Candida bombiphila strain MUCL 56142, fragments thereof or substances produced thereby.

The term “food composition”, also referred to as “feed composition”, as used herein generally refers to a combination of elements which can be ingested by an organism without causing harm to the organism. Preferably, at least a part of said elements are essential and/or non-essential nutrients which can be ingested and assimilated by an organism, in particular an arthropod, to produce energy, stimulate growth and/or maintain life. Even more preferably, said food composition is artificially produced.

In certain embodiments, the food composition as envisaged herein comprises Wickerhamiella yeast material, particularly Wickerhamiella bombiphila (or Candida bombiphila) yeast material, preferably a Candida bombiphila yeast strain deposited under accession number MUCL 56142 as further discussed herein.

In particular embodiments, the food composition for arthropods as envisaged herein comprises yeast material selected from the group of (i) living cells of said Wickerhamiella yeast; (ii) dead cells of said Wickerhamiella yeast; or (iii) a growth medium obtainable by first inoculating and incubating said Wickerhamiella yeast in a growth medium and subsequently removing the yeast cells (optionally after inactivation) from the medium.

In particular embodiments said food composition comprises between 10² and 10⁸, preferably between 10⁴ and 10⁵ yeast cells per mg or ml of the food composition. In particular embodiments, the food composition of the present invention is a liquid food composition comprising the yeast material as envisaged herein between 10² and 10⁸, preferably between 10⁴ and 10⁵ yeast cells/ml. In particular embodiments, the Wickerhamiella yeast cells, particularly the Wickerhamiella bombiphila (Candida bombiphila) yeast cells are alive within the food composition. In particular embodiments, the number of yeast cells in the compositions increases naturally over the time period that the composition is offered to the arthropod. In particular embodiments, the methods of the present invention involve offering the food composition as described herein to the arthropod for a period of between 1 and 30 days, such as for between 5 and 20, such as for 7 days. Within this time period, the concentration of yeast cells in the food composition may increase. For instance, in particular embodiments, the food composition comprises 100 yeast cells/microliter, and increases up to 60000 cells per microliter in 7 days. Candida bombiphila is a yeast and can be propagated according to any method for propagating yeasts known by the skilled person. For example, yeast cells can be grown in a liquid growth medium, such as Yeast Malt broth, preferably comprising Peptone, yeast extract, malt extract, glucose and agar, at about 24° C. for 24 to 48 hours standing on a shaking platform (e.g. 80-100 rpm). The concentration of yeast cells in a liquid medium, such as a liquid growth medium, can be measured using standard techniques, such as calculating the optical density of solution containing the yeast cells using a spectrophotometer or conducting cells counts of stained cells (the use of Methylene blue would allow to discern their viability, for instance) in an haemocytometer under the microscope at 40× magnification.

In certain embodiments, the food composition comprises at least one carbohydrate source. Carbohydrates form an essential part of the diet of many arthropods. Carbohydrates are mainly used to generate energy for muscular activity, body heat, and vital functions of certain organs and glands. Furthermore, carbohydrates, such as sugar, can act as feeding stimulants for arthropods. Additionally or alternatively, the carbohydrate source can function as a growth substrate for the yeast. Accordingly, in particular embodiments, the food composition comprises a sugar or a sugar alcohol as the carbohydrate source. In other particular embodiments, the food composition comprises nectar, honey or a substitute thereof as a carbohydrate source. The term “sugar” as used herein refers to soluble carbohydrates, such as glucose, fructose, galactose, sucrose, maltose, lactose, galactose, mannose, raffinose, dextrin, inulin, rhamnose, xylose, arabinose, trehalose or melezitose and compounds/products containing glucose, fructose, galactose, sucrose, maltose, lactose, galactose, mannose, raffinose, dextrin, inulin, rhamnose, xylose, arabinose, trehalose or melezitose, such as molasses, sugar beet sugar, cane sugar and hydrolysed starches but not limited thereto. Suitable sugar alcohols could include sorbitol and mannitol. In particular embodiments, the food composition comprises sucrose, maltose, glucose, fructose, dextrose or combinations thereof. For instance, 20 to 70% of the total amount of sugar is sucrose, 5 to 50% of the total amount of sugar is glucose and 5 to 50% of the total amount of sugar is fructose. Suitable sugars for the cultivation of yeast can include one or more of glucose, L-sorbose, D-ribose, and mannitol, glycerol and glucitol as sugar alcohols. It is noted in this regard that the food composition may additionally or alternatively comprise carbohydrates which are secreted by the yeast, such as but not limited to neo-kestose, 6-kestose, and bifurcose, and mannotriose.

In particular embodiments, the food composition comprising a Wickerhamiella yeast, particularly a Wickerhamiella bombiphila (Candida bombiphila) yeast as envisaged herein is a liquid, preferably aqueous, food composition. Preferably, said food composition as envisaged herein is a sugar solution comprising at least 10 wt % or at least 20 wt % of a sugar or sugar alcohol. More preferably, the (liquid) food composition has a sugar or sugar alcohol concentration ranging from 20 wt %, 25 wt % or 30 wt % to 50 wt %, 60 wt % or 70 wt %. The amounts of sugar(s) can be measured by methods known in the art such as high-performance liquid chromatography (HPLC) or a refractometer to detect sucrose equivalent in Brix. The sugar can be solubilised in, for example, water, milk or fruit juice, preferably water. In certain embodiments, at least 80 wt % or 85 wt %, more preferably at least 90 wt % or 95 wt %, such as 97 wt %, 98 wt %, 99wt % or 100 wt % of the nutrients in said food composition is a sugar or sugar alcohol as envisaged herein. In further particular embodiments, the sugar water has a concentration of 30%, wherein the sugar is made up of between 50-70% sucrose, between 10-20% glucose and between 10-20% fructose (based on dry weight). In more particular embodiments, the sugar in the sugar water is made up of 66% sucrose, 16.6% glucose and 16.6 fructose, on dry weight base.

In particular embodiments, the food composition comprising a Wickerhamiella yeast, particularly a Wickerhamiella bombiphila (Candida bombiphila) yeast as envisaged herein comprises a nitrogen source, preferably in addition to the carbohydrate source. Suitable nitrogen sources include pollen or suitable substitutes thereof, amino acids or proteins. Pollen can be naturally occurring, or a synthetic diet can be used as a pollen replacement, acting as a protein source (e.g. comprising at least 30% or at least 40% proteins). Pollen are a particularly suitable component of a food composition as envisaged herein: pollen comprises amino acids such as arginine, histidine, isoleucine, leucine, lysine, methionine, phenylalanine, threomine, tryptophane and valine, and contains a multitude of vitamins, minerals, and lipids. The protein content of pollens is preferably at least 10%, at least 20%, be provided in the form of, for instance, soy flower, torula yeast or brewer's yeast. Proteins can also be provided in the form of, for instance, soy flower, torula yeast or brewer's yeast.

The inventors have found that the yeast cells can survive for over 10 days in compositions which comprise sugar water with or without pollen. In particular embodiments, the food composition is a pollen ball made with pollen and sugar water, comprising Wickerhamiella, particularly Wickerhamiella bombiphila (Candida bombiphila). In particular embodiments, the pollen balls are made with 5-20%, preferably 10% sugar water comprising a Wickerhamiella yeast, particularly a Wickerhamiella bombiphila (Candida bombiphila). In further particular embodiments, the sugar water contains at least 100 yeast cells per microliter.

Optionally, other nutrients, including but not limited to lipids or fats, vitamins and/or minerals may be present in the food composition as envisaged herein as well. Vitamins can be any vitamin known by the skilled person, such as vitamins B, such as thiamine, riboflavin, nicotinamide (niacin, nicotinic acid), pyridoxine, pantothenate (pantothenic acid), folic acid, and/or biotin; and/or vitamin C (ascorbic acid), and/or vitamins D and/or vitamins E. Lipids or fats can be dietary lipids such as fatty acids, sterols and phospholipids. Lipids can be used for energy, synthesis of reserve fat, and for the functioning of cellular membranes. Minerals can be any dietary mineral known by the skilled person, including but not limited to sodium, potassium, calcium, magnesium, chlorine, phosphorus, iron, copper, iodine, manganese, cobalt, zinc, and/or nickel.

In certain embodiments, the food composition may meet the general nutritional requirements of arthropods, comprising nutrients, such carbohydrates (e.g. nectar, honey, sugar), proteins, lipids or fats, minerals, vitamins, and water. Accordingly, the food composition according to present invention may be used to provide arthropods with a fully nutritious, easily digestible, complex mixture of nutrients in amounts and proportions effective to support growth, development, maintenance, and reproduction. Thus, in particular embodiments, the food composition comprising a Wickerhamiella yeast, particularly the Wickerhamiella bombiphila (Candida bombiphila) yeast is specifically adapted to use as feed for arthropods.

In certain embodiments, the food composition as envisaged herein may comprise other organisms in addition to the Wickerhamiella yeast, particularly the Wickerhamiella bombiphila (Candida bombiphila) yeast as envisaged herein, however said yeast is the main organism, meaning that it is present in the largest numbers. In further embodiments, the food composition does not comprise any other organisms. The ingredients of the food composition as envisaged herein can be mixed according to standard methods known by the skilled person.

Advantageously, in particular embodiments, the food composition as envisaged herein may be sterilised or pasteurised to increase the shelf life as known and performed by the skilled person prior to the addition of the yeasts as envisaged herein or where the food composition is not envisaged to comprise live yeast cells, but rather comprises yeast fragments or products derived from yeast cells. This can be achieved by, for example, ultraviolet germicidal irradiation. Alternatively or in addition, the food composition may comprise a suitable preservative or antimicrobial agent for preventing spoilage of the food source composition and to enhance its shelf life. Suitable preservatives or antimicrobial agents are preferably not harmful for the yeasts in the food composition and are known in the art.

In alternative embodiments, the food composition comprises live yeast cells. Yeasts surviving in the gut of insects have been shown to provide decomposition products, B vitamins, sterols, or essential amino acids to their insect host (Jones 1984, Douglas 1998, Vega & Dowd 2005, Lee et al 2014). Therefore, it is reasonable to assume that the production of essential metabolites by yeast cells provides a fitness advantage for insects, more particularly for arthropods that rely on nutritionally poor or unbalanced substrates, such as floral nectar (Douglas 1989, Pozo et al 2014) or pollen (Roulston & Cane 2000). Accordingly, in particular embodiments, the yeast cells may help to digest the natural food source. Several lines of evidence indeed indicate that the nutrients from pollen are not promptly available and that some insects are unable to consume particular types of fresh unprocessed pollen. More particularly, for honeybees, it is known that they cannot digest starch well, such that they have difficulties digesting pollen types with a high starch content.

In further alternative embodiments, the food composition comprises both live yeast cells and dead yeast cells and/or fragments or products derived from yeast cells.

The food composition can be provided by any form of feeding device for arthropods known by the skilled in the art. The food composition as referred herein can be in a liquid, a paste or a dry/powder form, preferably a liquid form. In preferred embodiments, the food composition is a liquid food composition, in particular an aqueous food composition. Advantageously, a liquid food composition can be placed in a container and/or can be applied onto porous or fibrous items, such as a cotton ball or a capillary wick, which can be placed close to the nest or in the nest box and is available to the arthropod via capillary action. When the arthropods are pollinating insects, the food composition according to the invention can also be provided or sprayed on flowering crops. Preferably, the food composition is substantially odor-free, or if such food composition contains an odor, such odor should not be malodorous or repellent to arthropods.

The application further provides methods of making a food composition for the cultivation of arthropods as described herein. In particular embodiments, the methods comprise inoculating a sugar water composition with a Wickerhamiella yeast, particularly a Wickerhamiella bombiphila (Candida bombiphila). In particular embodiments, the methods comprise mixing pollen with sugar water comprising a Wickerhamiella yeast, particularly a Wickerhamiella bombiphila (Candida bombiphila) so as to obtain a concentration of said yeast of at least 100 cells/μl or 11 cells/μg or the equivalent thereof in fragments or products produced thereby. More particularly, this can encompass inoculating a concentrated yeast inoculum in sugar water. In particular embodiments, the sugar water is between 5-40%, such as 10-30%. In particular embodiments, the methods further comprise mixing the sugar water with pollen so as to obtain a pollen ball. Further embodiments of the methods are related to the food compositions as described herein.

Further, the inventors have isolated a specific Candida bombiphila/Wickerhamiella bombiphila strain from Bombus terrestris workers' guts. Advantageously, the provision of this specific yeast strain as envisaged herein to arthropods, more particularly bees, even more particularly B. terrestris, had a pronounced positive effect on the colony development and flight activity of the arthropods. These beneficial effects were absent in arthropods not receiving any Wickerhamiella yeast, particularly the Wickerhamiella bombiphila (Candida bombiphila).

Accordingly, further provided herein is a specific strain of Candida bombiphila/Wickerhamiella bombiphila yeast, more particularly the Candida bombiphila/Wickerhamiella bombiphila strain deposited on Jun. 21, 2016 in the BCCM/MUCL (Belgian Coordinated Collections of Micro-organisms (BCCM) Université catholique de Louvain, Mycothèque de I'Université catholique de Louvain (MUCL), Croix du Sud 2, box L7.05.06, 1348 Louvain-la-Neuve, Belgium) under the accession number MUCL 56142 (see Table A) or variants or derivatives thereof. Said strain or variants or derivatives thereof can be used to advantageously rear arthropods, preferably pollinating insects, and/or to improve the health, behaviour and/or general fitness of said arthropods, as further described herein.

TABLE A
Indications relating to deposited microorganism MUCL 56142

| | |
|---|---|
| Accession number given | MUCL 56142 |
| by depositary institution | |
| Identification reference | 170 |
| given by the depositor | |
| Name of depositary | Belgian Coordinated Collections of |
| institution | Micro-organisms (BCCM)/Mycothèque |
| | de l'Université catholique de |
| | Louvain (MUCL) |
| Address of depositary | Mycothèque de l'Université |
| institution | catholique de Louvain Croix du |
| | Sud 2, box L7.05.06 B-1348 |
| | Louvain-la-Neuve Belgium |
| Date of deposit | Jun. 21, 2016 |
| Name of depositor | Jean-Marc Vandoorne-Feys - CEO |
| | of Biobest Belgium |
| Address of depositor | Ilse Velden 18 |
| | 2260 Westerlo |
| | Belgium |

As referred to herein, the term “variants” refers to microbial variants such as mutational, insertional, and deletional variants of Candida bombiphila/Wickerhamiella bombiphila MUCL 56142 as well as microbial variants having a whole genome sequence identity of at least 90%, more preferably at least 95% and for instance at least 96%, 97%, 98%, 99% or 99.9%.

The following examples are provided for the purpose of illustrating the present invention and by no means are meant and in no way should be interpreted to limit the scope of the present invention.

### EXAMPLES

In this section, the yeast species, currently known as Wickerhamiella bombiphila (de Vega et al, 2017, FEMS Yeast Research, Volume 17, Issue 5, 1 Aug. 2017) is primarily referred to by the previously accepted “Candida bombiphila”.

### Example 1: Isolation of Candida bombiphila, and Taxonomic Information & Ecology

The Applicants have isolated a new C. bombiphila strain from a B. terrestris worker's gut. This worker originated from a colony started by a wild queen collected in Heverlee, Belgium, which was allowed to start a colony in the lab. It was the fastest growing strain of the species they encountered. The Applicants have deposited this C. bombiphila strain at the Belgian Coordinated Collections of Micro-organisms (BCCM)/Mycothèque de I'Université catholique de Louvain (MUCL) with accession number MUCL 56142 (further details on the strain deposit are indicated above in Table A).

Candida bombiphila was described in 2004 by Brysch-Herzberg & Lachance (Int J Syst Evol Microbiol 54 (2004) 1857-1859), based on the information of two strains. The type strain was deposited in the Yeast Division of the Centraalbureau voor Schimmelcultures, Utrecht, the Netherlands, as strain CBS 9712T (=NRRL Y-27640T=MH268T). It was isolated from the proboscis of a Bombus terrestris queen in early spring. In fact, the authors found C. bombiphila in only one bumblebee sample out of 135 (on the proboscis), whereas 103 samples of the 135 contained yeasts. A second strain, CBS 9713(=X316.5), was isolated from the honey provision in a nest of Bombus pascuorum bees in the summer. Both strains were isolated in the New Botanical Garden of Philipps University, Marburg, Germany. Although this seems to suggest that the habitat of the species is to be bumblebees and their honey provisions, more recent analyses of bumblebee specimen, and their food sources such as floral nectar, did not record this species. Ten years following the recording of the aforementioned German strains there was just one further sequence submitted with that affiliation by 15 Sep. 2016 from which the species was described. The 3^{rd} isolate (IMB11L4) comes from an insect as host, collected by Gouliamova, D. E., with regard to the unpublished paper “Biodiversity of yeasts in selected Bulgarian ecosystems”, submitted in 2013. In addition, Brysch-Herzberg (FEMS Microbiology Ecology 50 (2004) 87-100) concluded that no conclusion could be drawn about the ecology of C. bombiphila.

This is confirmed by the Applicant's own experiments. A total of 31 and 43 bumblebee queens (B. terrestris) were collected from the wild in spring of 2014 and 2016, respectively. The gut, crop, and proboscis of each bee were plated separately on YGC (Yeast Glucose plus Chloramphenicol) agar plate. Subsequently, the Applicants isolated all distinct cultured (i.e. live) yeasts and identified them to species level using Sanger sequencing and BLAST with a 99% identity. The Applicant's analyses showed that the yeast species Candida bombiphila was quite uncommon in wild bumblebees, as they just were isolated in 4% and 13% of the queens (all substrates included) in the 2014 and 2016 respectively, and in total they accounted for 5% of the gut-isolated yeasts.

Even though this yeast species might inhabit the tongue of the bumblebee and consequentially could colonize nectar, they were never isolated from this substrate, even though this has been thoroughly studied worldwide. In a recent review of all published records of nectar inhabiting microorganisms, C. bombiphila is not listed as nectar-inhabiting species (Pozo, M. I., Lievens, B., & Jacquemyn, H. (2014). Impact of microorganisms on nectar chemistry, pollinator attraction and plant fitness. nectar: production, chemical composition and benefits to animals and plants. Nova Science Publishers, Inc., New York). The relatively recent description (2004) of the taxa would not explain this knowledge gap, as most of the research in nectar inhabiting microbes have been carried out in the last decade (Pozo et al, 2014).

In parallel with those surveys, the Applicants have carried out surveys using the above described methodology to assess the gut flora of indoor-reared bumblebees and more particularly, if C. bombiphila could be transmitted from a source mother queen to subsequent generations and/or after hibernation of daughter queens. The Applicant's results show that this species was completely absent in commercially reared bumblebees (data not shown). According to Bab'eva and Chernov (2004; Biology of Yeasts, KMK, Moscow), yeasts are common inhabitants of the guts of invertebrates, but yeast symbionts are lost during subsequent generations of artificial rearing.

In addition, the Applicant carried out a separate experiment in which 10 bumblebee queens were actively fed on a weekly basis with living C. bombiphila growing in the sugar water. These 10 bumblebee colonies were allowed to complete the cycle in the standard commercial rearing conditions, before the preponderance of C. bombiphila in proboscis, crops, and guts was tested for several colony stages and individuals. Individuals tested included the founder queen, workers, and newly produced queens. The latter were tested both before and after the hibernation period. The Applicant's results unambiguously show that, from a sample of 2 daughter queens per nest that were processed before hibernation, none of them had this species. Interestingly, the same result was obtained for a sub-sample of 20 bumblebee queens that were processed after the hibernation period.

The D1/D2 sequence of the large-subunit rDNA of the type strain (CBS 9712T, collected from B. terrestris tongue) differs from the most closely related species, W. domercqiae, by 57 substitutions and three gaps. Strain CBS 9713 (AJ620186) differs from the type strain by one substitution and one gap. Kurtzman et al. [(Kurtzman et al. (1998) 73: 331. doi:10.1023/A:1001761008817) showed that in most cases distinct species differ by 1% or more in these sequences.

Multi-gene sequence analysis, such as for the Saccharomycetaceae (Kurtzman, & Robnett, (2003). FEMS Yeast Research, 3(4), 417-432), has shown that circumscription of yeast genera from phenotypic characters is often not congruent with their delineation based on molecular phylogenies. In contrast, the classification of asexually reproducing yeasts was rarely addressed in molecular phylogenetic studies because their inclusion in Candida and other anamorphic genera satisfied the requirements of longstanding rules under the International Code of Botanical Nomenclature. However, a recent change in the rules for naming pleomorphic fungi now requires that a fungal species or higher taxon be assigned only a single valid name under the new Code [International Code of Nomenclature for algae, fungi, and plants (Melbourne Code) (McNeill, et al. (2012). International Code of Nomenclature for algae, fungi, and plants (Melbourne Code). Regnum vegetabile, 154(1), 208)]. As a consequence, the current species of Candida and other asexual yeast genera must undergo revision to make genus membership consistent with phylogenetic affinities. In the case of species that currently possess both anamorph and teleomorph names, a decision will be required regarding which name to retain. In the previous system, the teleomorph name had priority, but the new Code places both names on an equal footing and reaffirms instead the principles of historical priority and common usage. According to Daniel et al. (Daniel, H. M., Lachance, M. A., & Kurtzman, C. P. (2014). On the reclassification of species assigned to Candida and other anamorphic ascomycetous yeast genera based on phylogenetic circumscription. Antonie van Leeuwenhoek, 106(1), 67-84) Candida bombiphila should move to the Wickerhamiella genus, namely then W. bombiphila. Indeed, recent research based on the DNA-based phylogeny of the species suggests that the species formerly known as “Candida bombiphila” should now be renamed as Wickerhamiella bombiphila, (de Vega et al, 2017).

### Example 2: Candida bombiphila (Living Cells) and its Effect on Bumblebee Health, Fitness, Behavior & Colony Development

Materials and Methods

To investigate the impact of C. bombiphila yeast, more particularly the C. bombiphila strain MUCL 56142 on bumblebee hive development and fitness, bumblebees received a diet comprising said C. bombiphila yeast. A control group received the same diet without the C. bombiphila yeast. For each treatment ten different nests were used as replicas. For all experiments, the earth bumblebee (Bombus terrestris) was used as study organism. To mimic their natural habitat (underground), bumblebees were held under a temperature of 29° C., a relative humidity of 60% and in a dark room. At the beginning of the experiment, 60 artificially reared mother queens were transferred to sterile artificial nest boxes and fed just for two days with a sugar solution (Biogluc®Biobest). Previous research has shown that post-overwintering queens that were kept in an artificial rearing program were devoid of yeasts. To decrease queen mortality, each queen was provided with a worker that was transferred as pupae to a sterile container, and then taken to the nest after emergence. This step ensures that workers are devoid of yeasts as well. During the whole experiment, bumblebees received pollen as a protein source. This pollen was deep frozen and irradiated with gamma irradiation at a minimum of 13 kGy and a maximum of 40 kGy to ensure that it was also devoid of micro-organisms. The effect of the yeast on colony development was assessed by evaluating the number of egg cups, the date when the first egg was produced, larval stages (first and second instar: L1-L2, and third and fourth instar: L3-L4), the number of workers, number of males and how many bees are dead (male, female, queen).

Yeast Preparation

The first step of the experiment consisted of inoculating the yeast strain from the primary or mother plate on Chloramphenicol Glucose Agar (YGC) plates to separate colonies. The agar was composed of the following components: 1.0% Glucose, 0.5% Peptone, 0.3% Malt extract, 0.3% Yeast extract, 2.0% agar, and 0.01% Chloramphenicol. Afterwards, the plates were inoculated at 24° C. for ±2 days so they could grow sufficiently.

In a second step, yeast cells were inoculated from the YGC plates into 5 ml of Yeast Malt (YM, 0.5% Peptone, 0.3% Yeast extract, 0.3% Malt extract, 1% Glucose, 2% agar) broth using an inoculating loop. After scraping a few cells off the source plate, the inoculating loop was scratched against the inside of the test tube. After inoculation, the test tubes were incubated at 24° C. for 48 hours standing on a shaking platform (80-100 rpm). By constantly rotating the sample tubes, the liquid was always in contact with the air.

The bees were provided with a final density of 100 cells per microliter. This value was derived from the average number of cells that have been found per microliter of nectar in a sample of 60 different plant species growing in SE Spain (Pozo et al., In: Peck et al. Nectar: production, chemical composition and benefits to animals and plants. Nova Science Publishers, Inc. NY, 2015).

A concentrated yeast inoculum (OD=0.05) was added to a 30% sugar water composition (with the sugar made up of 66% sucrose, 16.6% Glucose and 16.6 Fructose, on dry weight base) so as to obtain a concentration of 100 cells/μl. For a container of 20 mL we added 100 microliters of the inoculum. Control treatment was supplemented with the same volume of sterile YMB. Because yeast will deplete sugars in the containers and thereby may affect bee fitness, a second container with sterile sugar water at 50% ad libitum (with the sugar made up of 66% sucrose, 16.6% Glucose and 16.6 Fructose, on dry weight base) was placed next to the first container. Those solutions were taken by the bees by using a capillary wick connected to the nest area.

In a further experiment, the yeast broth was added to the sugar water plus (with the same number of cells) and mixed with the pollen, in a percentage of 10% w/w basis with respects to the pollen content.

It is noted that after 10 days, the yeast cells remained totally viable, such that these could also be used for administration to the bumblebees.

Assessing Hive Performance

Bumblebee Hive Development

To investigate how fast a colony grows and how growth was affected by the presence of the yeast, the number of egg cups were counted, the date when the first egg cup was produced was determined, the larval stages (L1-L2, L3-L4) were assessed, and the number of workers, number of males and the number of dead bees (male, female, queen) were counted on a weekly basis. This was done during 9 consecutive queens after placing the mother queen.

Data Analysis

To investigate whether the presence of yeast cells affected hive development, a mixed model analysis of variance was used. Hive development was assessed using three different variables, namely colony size, brood, and the number of predicted workers. For each dependent variable, treatment was used as a fixed effect and colony nested within week was used as a random variable, to state that repeated measurements were done for each colony. Prior to running the mixed models, lack of normality of the data was checked for each variable and Poisson was consistently chosen as most suitable distribution. Afterwards, post-hoc comparisons were performed to investigate which treatments had a similar impact on colony size, brood or the number of future workers. Before hives reached the “competition point”, in week 7 (8 weeks after queen placement), the number of predicted workers (actual workers plus pupae) was compared between treatments by using a generalized linear model, adding the position of the colonies in the climatic chambers as additional predictors.

The Use of Candida bombiphila to Enhance Arthropod Health

Live yeast Candida bombiphila (Cbh) (isolated from the gut of Bombus terrestris) was tested for its growth-inhibiting effect on the bumblebee gut parasite Crithidia bombi. More particularly, this protozoan pathogen was isolated from the faeces of B. terrestris and cultured as described in Salathe et al 2012 (Salathe et al., PLoS ONE. 2012; 7(11):e49046). Both organisms were introduced together into a liquid medium in two concentrations, i.e. an initial cell density of 10 cells/μL and 100 cells/μL. The in vitro experiment was carried out at a controlled temperature of 27° C. under two different atmospheric conditions, namely standard aerobic conditions and microaerophilic conditions (4% CO₂) that approach the conditions in the bumblebee gut. In order to assess normal cell death in the chosen medium and under chosen culturing conditions, we also inoculated liquid medium with either the Crithidia or the yeast separately, again in the same two concentrations. After a two-day incubation period, we identified the proportion of live and dead cells of both micro-organisms by using a specific staining method that stains dead cells only. Cells were fixed during a formaldehyde fixation step, so that the number of stained (dead) and unstained cells (live) could be counted in a Neubauer counting chamber at a later time.

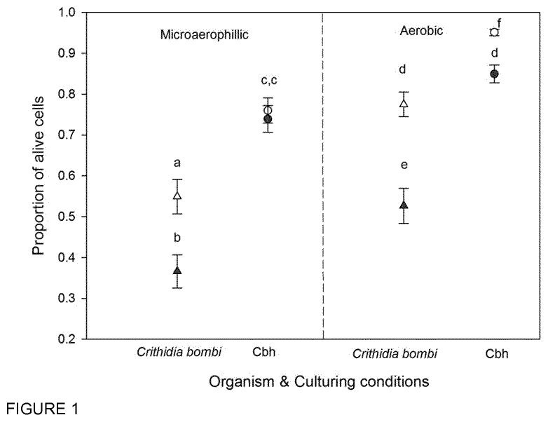

The results indicate that the survival of the parasite Crithidia bombi was significantly reduced when the yeast Candida bombiphila was added to the liquid medium, and this result was consistent under both atmospheric conditions (aerobic: odd's ratio=0.32, Z=−4.6203826, P=0.0001, microaerophilic: odd's ratio=0.47, Z=−3.046, P=0.048, FIG. 1). Interestingly, cell death of the yeast itself was low in all treatments. Under microaerophilic conditions, there was no significant decrease in the survival of yeast cells in the mixed-species-treatment compared to the yeast-only control, and yeast cells survived significantly better than Crithidia cells in the mixed-species-treatment (odd's ratio=4.91, Z=36.26, P<0.0001). Under aerobic conditions, however, there was a small decrease in yeast cell survival in the mixed-species treatment compared to the control (odd's ratio=0.29, Z=−5.14, P<0.0001) but Crithidia cell death was still much greater than yeast cell death (odd's ratio=5.06, Z=51.49, P<0.0001). Together, these results indicate a significant negative effect of the yeast Candida bombiphila on the survival of the gut parasite Crithidia bombi.

The Use of Candida bombiphila Living Cells to Enhance the Activity of Arthropods

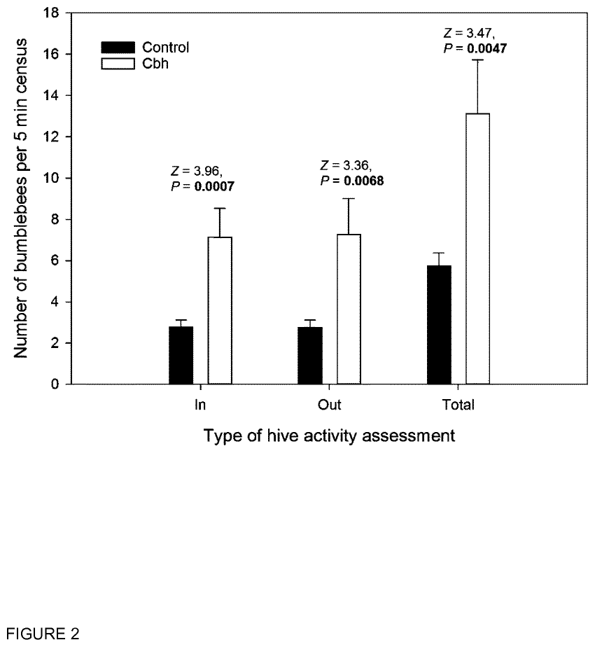

In this experiment, the flight activity of B. terrestris hives was followed up after feeding Candida bombiphila to the colonies throughout their entire development. The colonies were fed with a standard diet consisting of ad libitum sterilized honeybee-collected pollen and 50% sugar water. Next to this standard diet we provided 10 colonies daily with 30% sugar water solution containing 100 cells/μL of C. bombiphila, and as a control, 10 colonies were provided daily with a 30% sugar water solution+yeast malt broth (YMB). The solutions were administered in small vials attached to the walls of the brood box, and they were refreshed daily to avoid contamination (in the case of the yeast this means that we administered fresh one-day-old cells daily). When the hives reached the age of nine weeks, we selected 4 hives of each treatment to follow up their activity in the field. This selection was made based on a comparable number of workers and brood, the presence of a live mother queen, and the absence of any males. The hives were placed randomly in a flowering field and right before placing them outside, they were provided with one small bottle containing 20 mL of 30% sugar water solution with the C. bombiphila yeast (100 cells/μL) or without yeast for the control colonies. Subsequently, flight activity was assessed by carrying out counts of in- and out-flying workers. Counts were carried out on four different days with two 5-minute-rounds of counting each day. The results of this experiment show that feeding hives with a sugar water solution containing live Candida bombiphila cells increased flight activity of workers significantly when compared to hives that were not fed with the yeast. This effect was consistent for the number of workers flying into the hive and out of the hive (FIG. 2).

The Use of Candida bombiphila Living Yeast Cells to Optimize Arthropod Rearing

In this experiment, B. terrestris colony development was followed over a 12-week-period. Twenty colonies were kept under standard climatic conditions (28° C. and 60% relative humidity) and fed ad libitum with sterilised honeybee-collected pollen and a 50% sugar water solution. Next to this diet, 10 colonies received 30% sugar water containing 100 cells/μL of Candida bombiphila (+yeast malt broth), and the other 10 colonies received the same 30% sugar water solution (+YMB) without the yeast. This solution was administered in a small container underneath the brood box, and it was refreshed weekly (allowing the yeast to grow over a one-week-period). The larval developmental time was assessed by tracking the timing of first egg-laying, first development into pupae and first emergence of adults. During the first eight weeks, the number of workers, pupae, larvae, dead larvae, and eggs were counted weekly. At the end of the trial, after 12 weeks, the total number of sexuals (males and queens) were counted as well, as this is a parameter for colony fitness.

The results show that the administration of live Candida bombiphila has a significant positive effect on different parameters of colony development in B. terrestris.

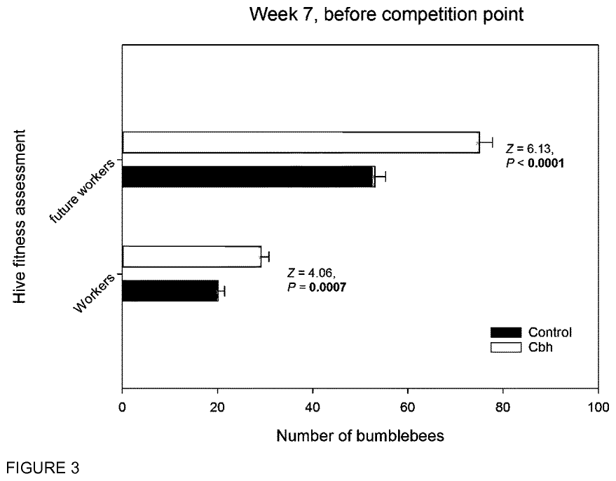

The results of counts after an eight-week-developmental period show that there is a significant increase in the number of produced workers when compared to control colonies that did not receive the yeast treatment (FIG. 3). This effect is also apparent when considering the number of pupae and workers summed up together, which is the predicted number of workers in one week time.

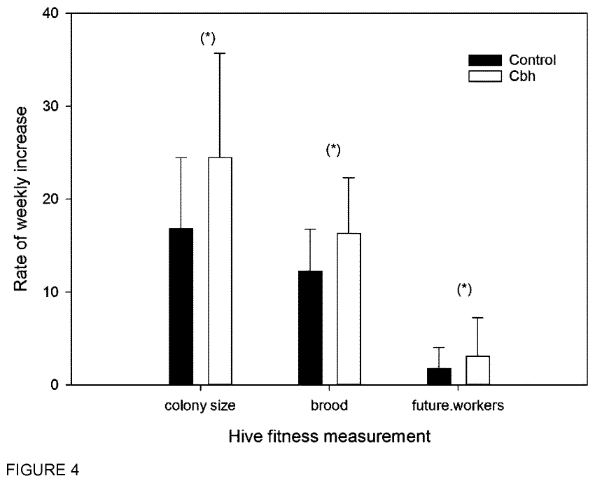

The fact that there are more predicted workers at a certain point in time compared to the control treatment indicates that colonies fed with C. bombiphila develop faster. The rate of the weekly increase in colony size (all developmental stages summed up), amount of brood (eggs and larvae), and future workers was also higher in the C. bombiphila treatment compared to the control (FIG. 4)

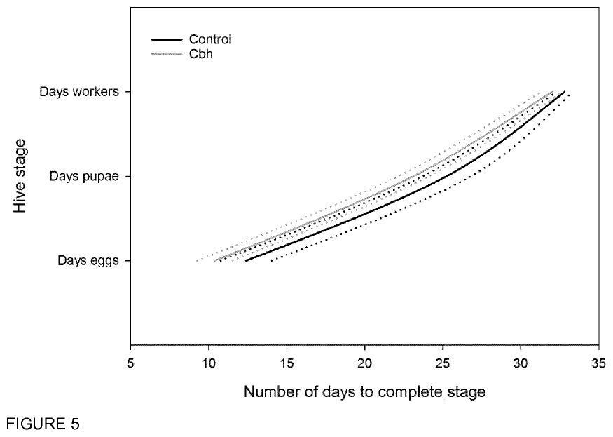

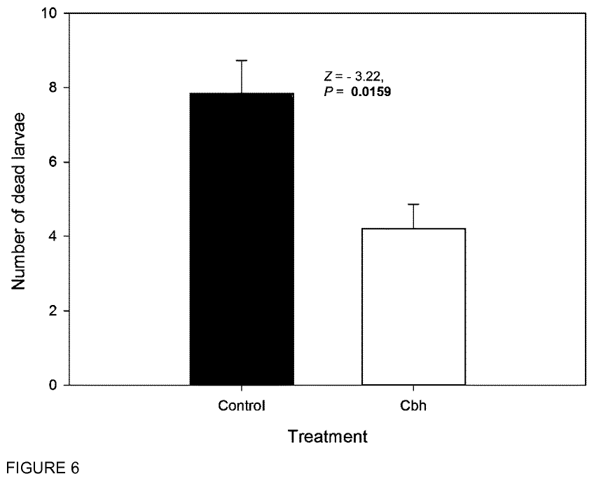

A more rapid colony development could imply a higher egg laying rate, a faster larval development and/or a higher larval survival. We can demonstrate that administration of C. bombiphila had at least two of these effects. First, the timing to reach the different developmental stages is slightly faster when C. bombiphila was administered (FIG. 5), but this effect was not significant. Second, the larval mortality was significantly reduced by the administration of C. bombiphila (FIG. 6).

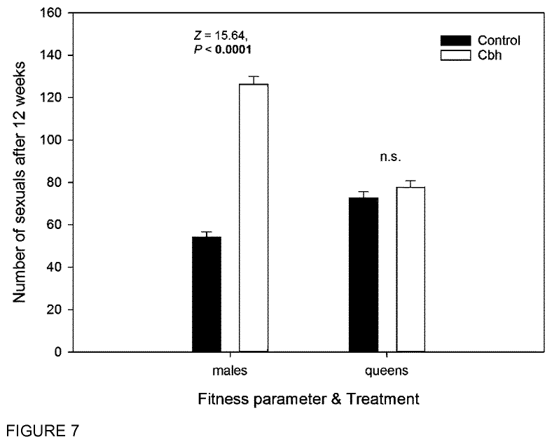

Administration of C. bombiphila also had a significant positive effect on the number of produced sexuals after a 12-week-period. This effect was apparent in the number of produced males but the effect was not significant for the number of produced queens (FIG. 7).

Similar experiments for assessing the rearing of arthropods in the presence or absence of C. bombiphila are performed with a predatory mite species, and a hoverfly.

Strain Specificity

The Applicants have tested the effect of two different strains of C. bombiphila on colony development and flight activity.

The first strain was a C. bombiphila strain that had previously been isolated from the proboscis of a wild B. terrestris queen in Germany and was deposited in the Yeast Division of the Centraalbureau voor Schimmelcultures, Utrecht, the Netherlands, as strain CBS 9712T (=NRRL Y-27640T=MH268T) (Brysch-Herzberg M. and Lachance M. (2004) Int J Syst Evol Microbiol, 54, 1857-1859, DOI 10.1099/ijs.0.63139-0). The Applicants obtained said C. bombiphila type strain CBS 9712T (‘Type strain’ henceforth) from the CBS-KNAW Fungal Biodiversity Centre, an institute of the Royal Netherlands Academy of Arts and Sciences.

The second strain is the C. bombiphila strain deposited by the Applicants at the Belgian Coordinated Collections of Micro-organisms (BCCM)/Mycothèque de I'Université catholique de Louvain (MUCL) with strain number MUCL 56142 (further details on the strain deposit are indicated above).

The Applicants tested both strains and compared their effect on bumblebee colony development and flight activity. Both strains were cultured and inoculated in the food source for B. terrestris bumblebees and the experiment was carried out as described in the materials and methods section above.

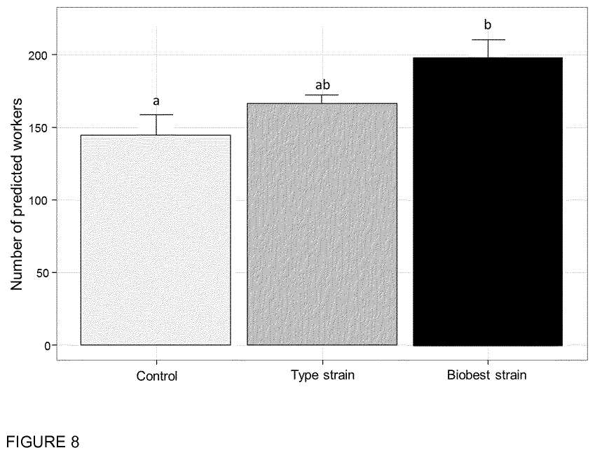

The Applicants results show that the MUCL 56142 strain had a strong positive effect on colony development, compared to a control treatment that did not contain any yeasts in the sugar water. The type strain CBS 9712T also positively affected colony development compared to the control but to a lesser extent than the MUCL 56142 strain. In particular the number of predicted workers was on average about 198.5±29.5 (SD) for the MUCL 56412 strain, while it was on average 166.2±16.2 (SD) for the Type strain CBS 9712T and on average 144.2±41.2 (SD) for the control (FIG. 8).

These results suggest a possible positive effect of both strains on the number of future workers that was produced within a time period of nine weeks (GLM, N_{colonies}=10), though this positive effect was only statistically significant for the MUCL 56142 strain. Additionally, the MUCL 56142 strain also had a positive effect on the total colony size (=all individuals and developmental stages of the colony summed up) when compared to the control treatment and within the time period of nine weeks.

Furthermore, the Applicants tested the effect of both strains on flight activity of hives that were fed with the yeast solutions throughout their entire development and compared this to hives that were fed with a control solution without any yeast added to it. The Applicants results show that both tested strains had strong positive effect on flight activity when hives were placed in the field and when compared to the control treatment without yeast (GLM with the number of workers present at start of the trial included as a covariate, N_{colonies}=4). More particularly, the MUCL 56142 strain showed a model-adjusted mean of flight activity of 2.35 and the Type strain CBS 9712T showed a model-adjusted mean of flight activity of 2.3, while the control only showed a model-adjusted mean of flight activity of 2.1 (data not shown).

Preference Tests

A behavioral test was performed to compare the attractiveness of a C. bombiphila treatment when added to artificial nectar, compared to control conditions in which no yeasts where present. The control diet was 30% sugar water (⅔ Sucrose (S), ⅙ Fructose (F), ⅙ Glucose (G)) plus sterile Yeast Malt Broth (YMB) in a proportion of 50 microliters per each 1 ml of sugar water. The C. bombiphila comprising diet was the same as the control diet, wherein the YMB was incubated with the yeast (final concentration 100 cells/u1).

One indoor-reared B. terrestris colony of 10 weeks, which had not been in contact with micro-organisms in their food before, was chosen at the Biobest bumblebee production. One day before the start of the trial, the Biogluc® access of the hive was turned off to let them starve and prepare them to forage. The colony was then opened inside a greenhouse equipped with an experimental flying arena. This arena contained 16 robotic flowers, plus a yellow plastic ring as landing platform, divided in 4 groups. This system mimics the system described by Kuusela and Lamsa (Ecology & Evolution, 2016, doi:10.1002/ece3.2062). The system includes a control unit, separate flowers, and a personal computer. The function of the control unit is to handle the electronics of the flowers, collect data from them, and to send data to the computer. The robotic, artificial flowers themselves contain an infrared (IR) sensor and associated electronics, and an electromechanical device (servo) that offers a small, precise amount of sugar solution (referred from hereafter as “artificial nectar”) from a reservoir. The computer runs software that controls the refilling rate and data collection via the control unit. The rationale of the system is that the entrance of the bumblebees in the artificial flowers causes a voltage drop that is registered by a custom-made Java interface. Date and time of visitation, flower identity, and probing time for each effective visit is registered. Flower depletion in wild flowers is mimicked by setting an automatic refilling period of 10 minutes, so that the sugar water (artificial nectar) is not continuously offered and visitation rate mimics probing time in wild flowers.

The trial was composed of 16 artificial flowers with half of the flowers containing the control and half the C. bombiphila treatment. Over 2 days, the number of effective visits to artificial flowers containing control artificial nectar (without yeasts) and to artificial flowers containing C. bombiphila yeast cells was monitored, as well as the total time spent consuming nectar from each flower type.

The results showed that naïve bumblebees preferred flowers that contained C. bombiphila, as they received both a higher number of visits, in total, and those visits lasted longer (FIG. 15).

### Example 3: Administration of Candida bombiphila Via Pollen

Example 2 demonstrated that adding C. bombiphila via sugar water increases the fitness of bumblebee colonies reared in captivity, which are devoid of this yeast species. In the present example, it was assessed whether this effect can also be obtained when administering the yeast (living yeast cells) via pollen. Pollen were prepared by adding 20% (w/w basis) of a 40% concentrated sugar water (⅔ Sucrose, ⅙ Glucose, ⅙ Fructose), knead into sausages. This composition was provided to the developing bumblebee colonies. The food was refreshed weekly, although the yeast stayed viable for 4 weeks inside the pollen (results not shown).

The MUCL 56142 C. bombiphila strain was suspended in Yeast Malt broth. An inoculum of A600=0.25 was used, which was later suspended in a proportion of 5 microliters per ml of 40% sugar water (⅔ Sucrose, ⅙ Fructose, ⅙ Glucose) without preserving agents. This protocol would ensure a dosage of 100 cells per microgram of pollen sausage, as was used in the sugar water administration in Example 2. A total of 20% of this mix of sugar water and Yeast Malt Broth inoculum was added to a certain weight of sterile (gamma-irradiated) honeybee collected pollen.

The performance of 15 colonies fed with this pollen mix was compared to 15 (different) control colonies, which did not receive the yeast strain. As a carbohydrate source, the colonies received Biogluc® ad libitum.

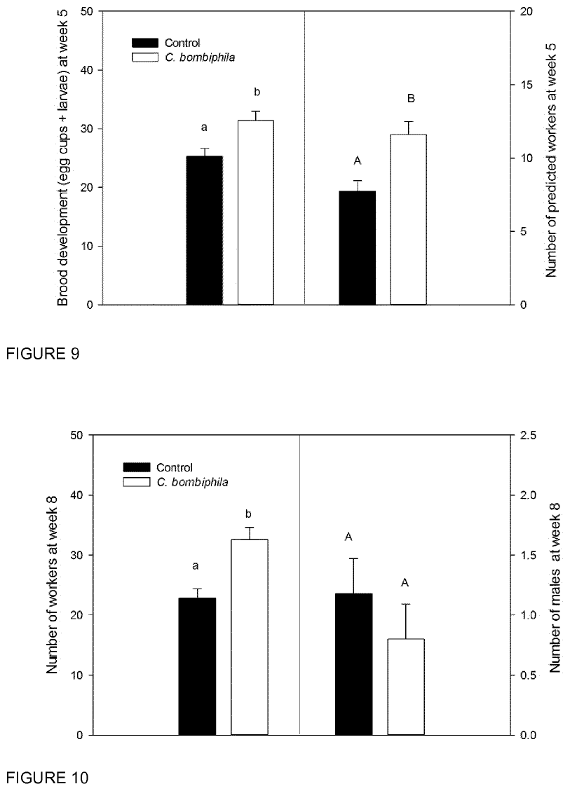

First workers appeared after 36.4+−1.6 (mean+−SE) days in control colonies and after 33.6+−1.5 (mean+−SE) days in colonies fed with pollen containing living C. bombiphila. At 5 weeks, colony development was significantly faster when colonies received the living C. bombiphila strain MUCL 56142 when administered via the pollen. This positive effect on development can both be seen on brood size (Sum of egg cups and larvae, FIG. 9, left panel) and the number of predicted workers at that time (FIG. 9, right panel).

At 8 weeks colony development, all aforementioned colony development parameters were assessed. The results show that addition of C. bombiphila to the pollen positively affected all colony development parameters that would affect the saleability of the colony and its future performance in the field. The number of emerged workers at 8 weeks was significantly higher in colonies that received pollen with C. bombiphila, i.e. 32.6+−2.0, than in control colonies that only reached 22.8+−1.6 workers at the same point in time (FIG. 10, left panel). The amount of males tended to be lower in the colonies supplemented with C. bombiphila, although this difference did not reach statistical significance at this time of colony development (FIG. 10, right panel).

Examples 2 and 3 thus demonstrate that the beneficial yeast can be provided via a liquid food compostion, i.e. a sugar solution or artificial nectar solution, or via a solid, pollen based food composition.

### Example 4: Candida bombiphila (Cells, Fragments, Produced Substances) and its Effect on Bumblebee Health, Fitness, Behavior & Colony Development

The use of Candida bombiphila (Cells, Fragments, Produced Substances) for Optimizing Arthropod Rearing

In this experiment, B. terrestris colony development was followed over a 16-week-period. A total of 60 colonies were kept under standard climatic conditions (28° C. and 60% relative humidity) and fed ad libitum with sterilized honeybee-collected pollen and a 50% sugar water solution (⅔ Sucrose (S), ⅙ Fructose (F), ⅙ Glucose (G)). Next to this diet, all colonies received a second diet as indicated in Table 1. As detailed below, this second diet comprised living yeast cells (treatment 1), dead yeast cells/fragments (treatments 2 & 3, with or without inoculation medium, respectively) or yeast metabolites/fragments (treatment 4—as the inoculation medium wherein the yeast was cultivated, but subsequently inactivated and filtered)

Control colonies (N=12) received (ad libitum) 30% sugar water (same composition as the aforementioned 50%) plus sterile Yeast Malt Broth (YMB) in a proportion of 50 microliters per each 1 ml of sugar water. The remaining colonies (N=48) also received a second diet composition via a sugar water feeder (ad libitum), with composition as listed in Table 1. All sugar solutions were free of preserving agents, and were refreshed weekly. They were administered via two separate containers underneath the brood box.

TABLE 1
Experimental design for C. bombiphila (strain MUCL
56142) administration via sugar water composition

| | Initial | |
|---|---|---|
| Treatment | dosage | Second diet composition |
| Control | — | 30% sugar solution (⅔ S, |
| | | ⅙ F, ⅙ G) + 50 μl/ml YMB] |
| 1. living | 100 cells/μl | 30% sugar solution (⅔ S, |
| C.b. cells | | ⅙ F, ⅙ G) + 50 μl/ml |
| | | inoculated YMB] |
| 2. C.b. cells, | 100 cells/μl | 30% sugar solution (⅔ S, |
| inactivated | | ⅙ F, ⅙ G) + 50 μl/ml |
| after 3 days | | inoculated YMB - yeasts were killed by |
| | | heat shock after 3 days (OD = 0.05) |
| 3. Inactivated | OD = 0.05 | 30% sugar solution (⅔ S, |
| C.b. cells | | ⅙ F, ⅙ G) + 50 μl/ml YMB + |
| | | 0.3 ml of inactivated yeast pellet |
| 4. Inactivated | 100 cells/μl | 30% sugar solution (⅔ S, |
| C.b. cells | | ⅙ F, ⅙ G) + 50 μl/ml |
| (+filtered) | | inoculated YMB - yeasts were killed by |
| | | heat shock after 3 days (OD = 0.05) |
| | | & medium was filtered (0.25 μm) |

The larval developmental time was assessed by tracking the timing of first egg-laying, first development into pupae and first emergence of adults. At week 5 and week 10, the number of workers, pupae, larvae, dead larvae, and eggs were counted. At week 11.5, fully developed colonies (colonies with more than 80 workers) were selected per treatment and transferred into bigger boxes where they were allowed to develop further for another 5 weeks. At the end of the trial (16wk) all aforementioned parameters were assessed. In addition, the total number of sexuals (males and queens) were counted as well, as this is a parameter for colony fitness. The sum of pupae and workers, hereafter named as “predicted workers” is used as an additional variable to assess colony performance.

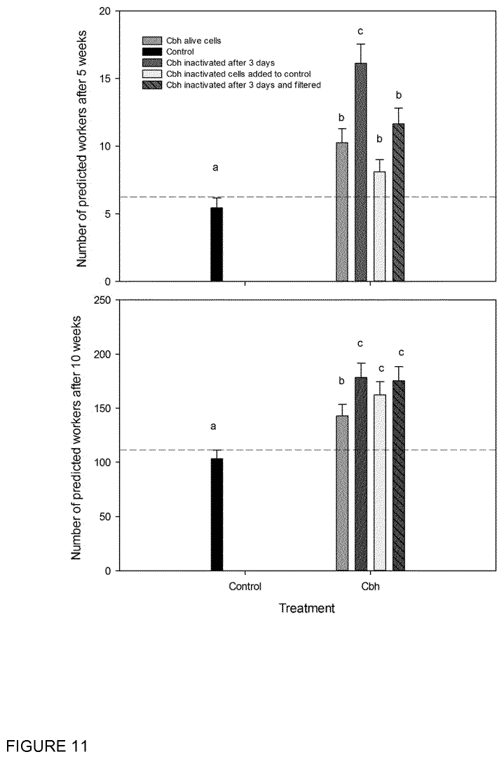

The results of counts after a five and a ten-week-developmental period show that there is a significant increase in the number of predicted workers when compared to control colonies that did not receive a yeast treatment, regardless of the way of administration (FIG. 11). This effect is also apparent when considering the actual number of workers at a given period (results not shown).

At 5 weeks, which reflects initial colony development and founding of the colony, the strongest positive effect on colony development was found when the medium with the inactivated cells (treatment 2) is administered (FIG. 11, upper panel). The count at 10 weeks shows that all treatments still perform significantly better than the control but that the addition of living cells (treatment 1) are slightly less positive than the other treatments. This may be due to the proliferation of the living yeast cells, which can reach high numbers and deplete the substrate while growing. These results also suggests that the effect can be optimized by administering inactivated cells once, which have been inactivated when they reach a plateau in their growth after 3 days. This ensures a yeast supplement with stable properties over time.

These results demonstrate that the beneficial effect of feeding C. bombiphila to insects can be obtained by different, non-exclusive, pathways:
the yeast may be administered under the form of living yeast cells (treatment 1). If administered as living yeast cells under the form of a medium wherein the yeast is cultivated, this treatment will comprise yeast metabolites as well;
the yeast or fragments thereof may be administered under the form of dead cells, either as the dead cells as such (without the medium wherein the yeast has been cultivated; treatment 3) or under the form of a medium wherein the yeast was first cultivated and subsequently inactivated (treatment 2)—without removal of the dead cells, or
the yeast, particularly yeast fragments or substances produced by said yeast may be administered under the form of a medium wherein the yeast cells were first cultivated and subsequently inactivated and removed, such as by filtration (treatment 4). Without wishing to be bound by theory, this will primarily provide yeast fragments and/or substances produced by the yeast and retained in the medium to the arthropod.

As demonstrated herein, all these treatments had a similar positive effect on colony development, resulting in a faster development with higher number of workers produced compared to the control treatment—i.e. in the absence of the C. bombiphila yeast, fragments thereof or substances produced thereby (FIG. 11).

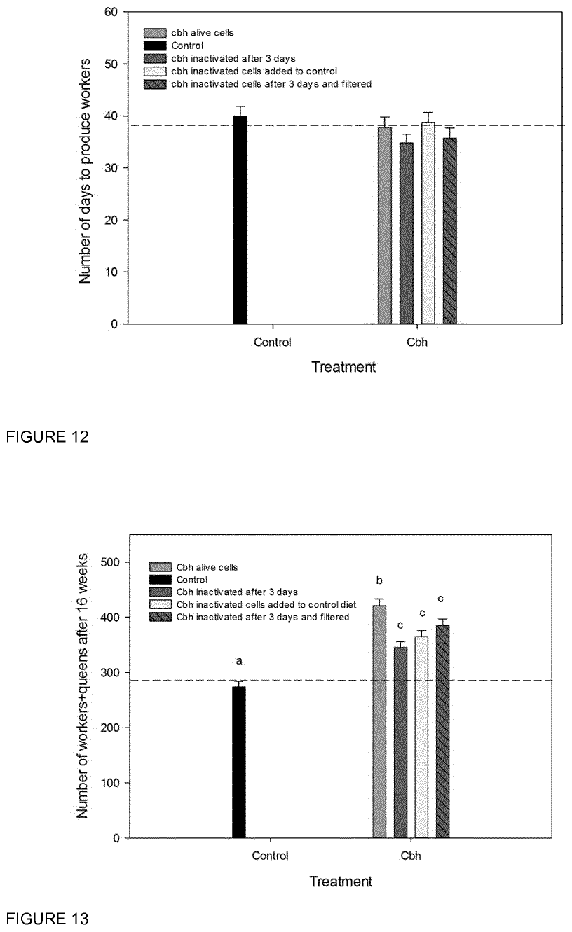

In addition, to the counts at certain time points (5 and 10 weeks), colonies were continuously monitored to record the first appearance of egg cups, pupae, and emerged workers. The results are represented in FIG. 12 and show that the administration of inactivated C. bombiphila (with or without cells present) led to a quicker appearance of workers (FIG. 12).

When colonies where allowed to develop further during a total 16-week-period, all the C. bombiphila administration treatments had a significant positive effect on the number of produced workers and queens (female fitness component) (FIG. 13).

Effect of Candida bombiphila (Cells, Fragments, Produced Substances) on Arthropod Behavior—Flight Activity

Five extra colonies were reared separately at Biobest NV in Belgium by using the aforementioned treatment combinations (N=5*5=25 colonies, see Table 1 for experimental settings). There was full brood development at all instances excepting one “control” and one “cbh inactivated after 3 days and filtered” treatments. Once the colonies reached above 120 workers, all colony development parameters were assessed (brood, pupae, workers . . . ) and colonies were then transferred to an apple orchard in Sint-Truiden, Belgium. Colonies were placed in blocks of 2, and those blocks were randomly scattered along 6 different rows of the orchard. All colony entrances were oriented to the South East, and hives were protected with a polysterene bee coat in view of the low temperatures and rainy conditions during early spring. We kept track of the flight activity by counting the number of workers flying in and out the hive during a period of five minutes. Those 5 minute censuses were conducted for all the colonies at 3 different timeslots (early morning, noon and afternoon) to account for overall flight activity throughout the day. Overall, hive activity was monitored for a total of 7 non-consecutive days that were divided in 2 consecutive weeks after placing the colonies in the field.

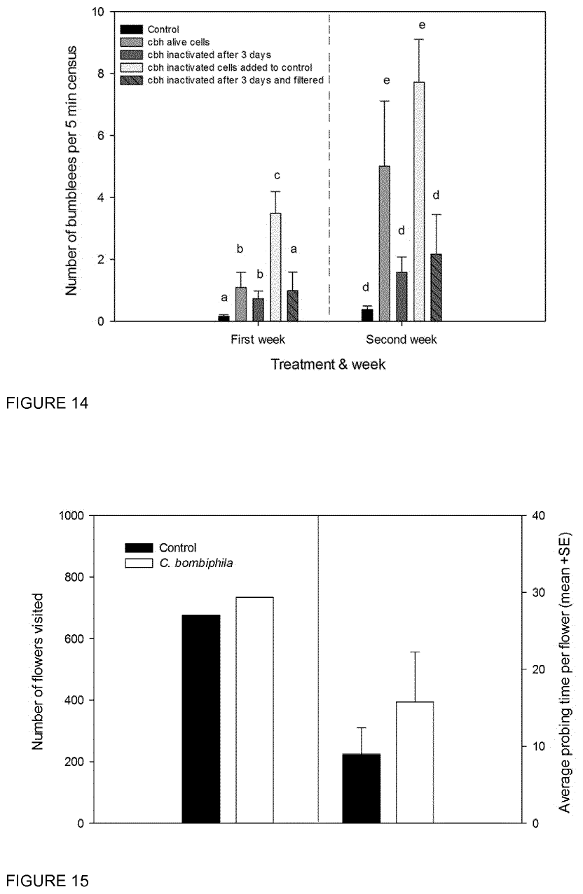

As the colonies receiving the “inactivated after 3 days” C. bombiphila treatment were more developed than the other treatments at the time they were placed, we introduced predicted workers (pupae+workers) as a covariate in the statistical model to correct for colony sizer differences and therefore calculate flight activity for colonies that would be ideally equal in size. The overall flight activity of the colonies (incoming plus outcoming bees per 5-minute census) was higher during the second week of the trial, coinciding with milder weather conditions. For a given week of monitoring, we consistently found that two different C. bombiphila administration treatments resulted in a significantly higher colony flight activity in the field, i.e. the administration of living cells in sugar water and the administration of inactivated cells in control sugar water (FIG. 14).

1. A method for rearing arthropods and/or improving the fitness of arthropods, comprising providing said arthropods with a Wickerhamiella yeast, fragments thereof or substances produced thereby.

2. The method for rearing arthropods according to claim 1, wherein said Wickerhamiella yeast is a Wickerhamiella bomblphila yeast.

3. The method for rearing arthropods according to claim 1, wherein said arthropods are colony-forming arthropods.

4. The method for rearing arthropods according to claim 3, wherein said method is a method for improving the development, size and/or fitness of a colony of arthropods.

5. A method for cultivating a fruit-bearing crop, comprising the steps of:
providing a flowering fruit-bearing crop;
providing a pollinating arthropod; wherein said arthropod is reared according to the method of claim 1; and
ensuring pollination of the flowering crop by the pollinating arthropod.

6. The method according to claim 1, for improving or enhancing the health and/or behaviour of arthropods.

7. The method according to claim 6 for improving immune functioning of arthropods, preferably wherein said Wickerhamiella yeast, preferably Wickerhamiella bombiphila decreases gut parasites such as Crithidia bombi.

8. The method according to claim 6, wherein said arthropods are pollinating flying insects.

9. The method according to claim 8 for improving flight activity.

10. A food composition for arthropods comprising sugar water and/or pollen, and a Wickerhamiella yeast, preferably Wickerhamiella bombiphila, fragments thereof or substances produced thereby.

11. The food composition according to claim 10, comprising at least 100 cells of said Wickerhamiella yeast per μl or per μg of said food composition.

12. The food composition for arthropods according to claim 10, further comprising one or more of the following: a carbohydrate source, preferably a sugar, or nectar or honey or a substitute thereof; a nitrogen source; vitamins; lipids or fats; minerals.

13. The food composition for arthropods according to claim 12, wherein said carbohydrate source is a sugar chosen from sucrose, glucose, maltose, dextrose, fructose, invert sugar, corn syrup or glucose syrup, and combinations thereof.

14. The method according to claim 1, wherein said Wickerhamiella yeast, preferably Wickerhamiella bombiphila is comprised within a food composition comprising sugar water and/or pollen.

15. A Wickerhamiella bombiphila or Candida bombiphila strain as deposited under the accession number MUCL 56142 at the BCCM/LMG culture collection, or variants thereof.

16. The method according to claim 1, wherein said Wickerhamiella yeast is a Wickerhamiella bombiphila strain as deposited under the accession number MUCL 56142 at the BCCM/LMG culture collection, or one or more variants thereof.

17. The method according to claim 1, wherein said arthropods are insects, preferably Hymenoptera.

18. The methods, the uses and/or the food compositions according to claim 17, wherein, wherein said Hymenoptera are Apocrita, preferably Apoidea, more preferably bees or bumble bees.
